(19) United States
(12) Patent Application Publication
(10) Pub. No.: US 2010/0010123 A1
(43) Pub. Date: Jan. 14, 2010

(54) ULTRAVIOLET ABSORBENT AND POLYMER MATERIAL CONTAINING THE SAME

(76) Inventors: Yoshihiko Fujie, Minami-ashigara-shi (JP); Takashi Hoshimiya, Minami-ashigara-shi (JP); Hisashi Mikoshiba, Haibara-gun (JP)

Correspondence Address: BIRCH STEWART KOLASCH & BIRCH, PO BOX 747, FALLS CHURCH, VA, 22040-0747, US

(21) Appl. No.: 12/499,493

(22) Filed: Jul. 8, 2009

(30) Foreign Application Priority Data
Jul. 9, 2008 (JP) 2008-179501

(51) Int. Cl. C08K 5/3445; C07D 231/34

(52) U.S. Cl. 524/106; 548/366.4; 548/367.1

(57) ABSTRACT

An ultraviolet absorbent, containing a compound represented by formula (1) or (2); and a polymer material containing the ultraviolet absorbent:

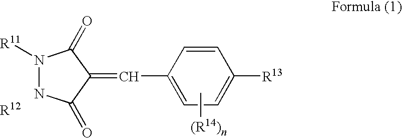

wherein R¹¹, R¹² and R¹⁴ each independently represent a monovalent substituent; R¹³ represents a hydrogen atom or a substituent having a Hammett substituent constant σₚ of −0.35 or more; and n represents an integer of 0 to 4; and

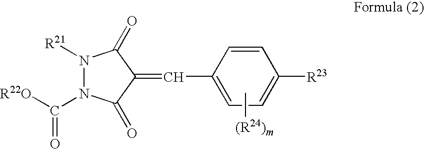

wherein R²¹ represents a substituted or unsubstituted alkyl group having 1 to 18 carbon atoms, or a substituted or unsubstituted phenyl group; R²² represents a substituted or unsubstituted alkyl group having 1 to 18 carbon atoms; R²³ represents a hydrogen atom or a substituent having a Hammett substituent constant σₚ of −0.35 or more; R²⁴ represents a monovalent substituent; and m represents an integer of 0 to 4.

### FIELD OF THE INVENTION

The present invention relates to an ultraviolet absorbent and a polymer material containing the same.

### BACKGROUND OF THE INVENTION

Ultraviolet absorbents have been used in combination with various resins for providing the resins with ultraviolet-absorptivity. Both inorganic and organic ultraviolet absorbents are used. The inorganic ultraviolet absorbents (see, for example, JP-A-5-339033 (“JP-A” means unexamined published Japanese patent application), JP-A-5-345639 and JP-A-6-56466) are superior in durability properties such as weather resistance and heat resistance. However, the freedom in selecting the compound is limited, because the absorption wavelength is determined by the band gap of the compound. In addition, there is no inorganic absorbent that absorbs the light in a long-wavelength ultraviolet (UV-A) range of 320 to 400 nm. And any such absorbent that absorbs long-wavelength ultraviolet would have color because it would have absorption also in the visible range.

In contrast, the freedom in designing the absorbent structure is much wider for organic ultraviolet absorbents, and thus, it is possible to obtain absorbents having various absorption wavelengths by designing the absorbent chemical structure properly.

Various organic ultraviolet absorbent systems have been studied, and for absorption in the long-wavelength ultraviolet range, it is conceivable either to use an absorbent having the wavelength of maximal absorption in the long-wavelength ultraviolet range or to use a high concentration of absorbent. However, the absorbents described in, for example, JP-A-6-145387 and JP-A-2003-177235 having the wavelength of maximal absorption in the long-wavelength ultraviolet range are inferior in light stability, and their absorption capacity declines over time.

In contrast, benzophenone- and benzotriazole-based ultraviolet absorbents are relatively superior in light stability, and increase in concentration or film thickness leads to relatively clean blocking of the light in the longer-wavelength range (see, for example, JP-T-2005-517787 (“JP-T” means published Japanese translation of PCT application) and JP-A-7-285927). However, when such an ultraviolet absorbent is applied as mixed with a resin or the like, the film thickness is limited to several tens of μm at the most. For utilizing the film thickness to block the light in the longer-wavelength range, it is necessary to add the ultraviolet absorbent to a considerably high concentration. In such a case, there were problems of precipitation of the ultraviolet absorbent and bleed-out during long-term use. In addition, among benzophenone-based and benzotriazole-based ultraviolet absorbents, there are some ultraviolet absorbents that may cause concern about skin irritation and accumulation in body.

Bulletin of the Chemical Society of Japan (1932), vol. 7, p. 45-49 and Journal of Chemical Physics (1950), vol. 18, p. 1307-1308 each describe a 4-benzylidenepyrazolidine-3,5-dion compound.

### SUMMARY OF THE INVENTION

The present invention resides in an ultraviolet absorbent, comprising a compound represented by formula (1):

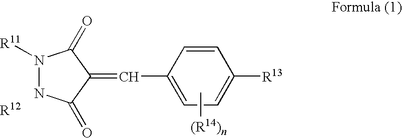

wherein R¹¹, R¹² and R¹⁴ each independently represent a monovalent substituent; R¹³ represents a hydrogen atom or a substituent having a Hammett substituent constant σₚ of −0.35 or more; and n represents an integer of 0 to 4.

Further, the present invention resides in an ultraviolet absorbent, comprising a compound represented by formula (2):

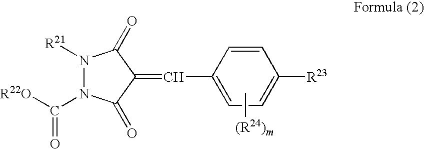

wherein R²¹ represents a substituted or unsubstituted alkyl group having 1 to 18 carbon atoms, or a substituted or unsubstituted phenyl group; R²² represents a substituted or unsubstituted alkyl group having 1 to 18 carbon atoms; R²³ represents a hydrogen atom or a substituent having a Hammett substituent constant σₚ of −0.35 or more; R²⁴ represents a monovalent substituent; and m represents an integer of 0 to 4.

Further, the present invention resides in a polymer material, comprising any one of the above ultraviolet absorbents, and at least one kind of polymer substance.

Furthermore, the present invention resides in a compound represented by formula (2):

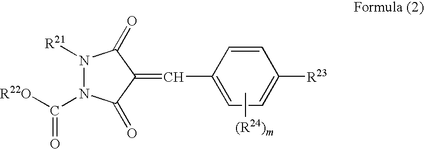

wherein R²¹ represents a substituted or unsubstituted alkyl group having 1 to 18 carbon atoms, or a substituted or unsubstituted phenyl group; R²² represents a substituted or unsubstituted alkyl group having 1 to 18 carbon atoms; R²³ represents a hydrogen atom or a substituent having a Hammett substituent constant σₚ of −0.35 or more; R²⁴ represents a monovalent substituent; and m represents an integer of 0 to 4.

Other and further features and advantages of the invention will appear more fully from the following description, appropriately referring to the accompanying drawing.

### BRIEF DESCRIPTION OF THE DRAWINGS

FIG. 1 shows spectra of the kneaded ultraviolet absorbent-containing polymer films (sample Nos. 301 and 302) prepared in Example 3.

### DETAILED DESCRIPTION OF THE INVENTION

The present inventors have found that it is possible to provide, by employing a compound having a specific chemical structure high in light fastness, a polymer material resistant to precipitation and bleed-out of the compound during long-term use, superior in long-wavelength ultraviolet absorption capacity, and superior in light fastness as it preserves the absorption capacity for an extended period of time. The present invention was made based on these findings.

According to the present invention, there are provided the following means:

[1] An ultraviolet absorbent, comprising a compound represented by formula (1):

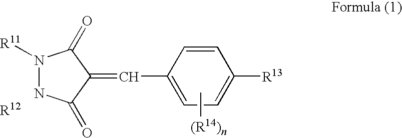

wherein R¹¹, R¹² and R¹⁴ each independently represent a monovalent substituent; R¹³ represents a hydrogen atom or a substituent having a Hammett substituent constant σₚ of −0.35 or more; and n represents an integer of 0 to 4.

[2] An ultraviolet absorbent, comprising a compound represented by formula (2):

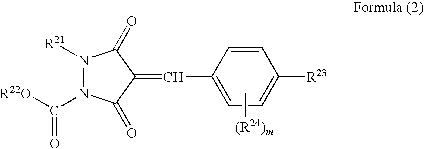

wherein R²¹ represents a substituted or unsubstituted alkyl group having 1 to 18 carbon atoms, or a substituted or unsubstituted phenyl group; R²² represents a substituted or unsubstituted alkyl group having 1 to 18 carbon atoms; R²³ represents a hydrogen atom or a substituent having a Hammett substituent constant σₚ of −0.35 or more; R²⁴ represents a monovalent substituent; and m represents an integer of 0 to 4.

[3] A polymer material, comprising the ultraviolet absorbent described in the above item [1] or [2], and at least one kind of polymer substance.

[4] The polymer material described in the above item [3], wherein the polymer substance is at least one kind of substance selected from the group consisting of acrylic acid-based polymers, polyester-based polymers and polycarbonate-based polymers.

[5] The polymer material described in the above item [3] or [4], wherein the glass transition point (Tg) of the polymer substance is −80° C. or higher and 200° C. or lower.

[6] The polymer material described in any one of the above items [3] to [5], wherein the polymer substance is a polyacrylate, a polycarbonate or a polyethylene terephthalate.

[7] The polymer material described in any one of the above items [3] to [6], wherein the polymer substance is a polyethylene terephthalate, and wherein the ultraviolet absorbent is contained in an amount of 0.1 mass % to 50 mass % with respect to 100 mass % of the polyethylene terephthalate.

[8] The polymer material described in the above item [7], which is prepared by melt-kneading of the polyethylene terephthalate and the ultraviolet absorbent at a temperature of 200° C. or higher.

[9] The polymer material described in any one of the above items [3] to [6], wherein the polymer substance is a polyacrylate or a polycarbonate, and wherein the ultraviolet absorbent is contained in an amount of 0.1 mass % to 50 mass % with respect to 100 mass % of the polyacrylate or polycarbonate.

[10] The polymer material described in the above item [9], which is produced by the steps of:

dissolving the polyacrylate or polycarbonate, and the ultraviolet absorbent in a solvent having a boiling point of 200° C. or lower; and

applying the obtained solution on a substrate.

[11] A compound represented by formula (2):

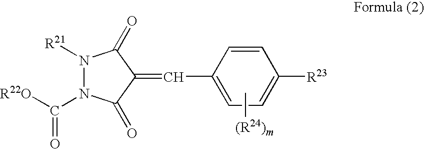

wherein R²¹ represents a substituted or unsubstituted alkyl group having 1 to 18 carbon atoms, or a substituted or unsubstituted phenyl group; R²² represents a substituted or unsubstituted alkyl group having 1 to 18 carbon atoms; R²³ represents a hydrogen atom or a substituent having a Hammett substituent constant σₚ of −0.35 or more; R²⁴ represents a monovalent substituent; and m represents an integer of 0 to 4.

[12] The compound described in the above item [11], wherein R²³ is a hydrogen atom, a substituted or unsubstituted alkyl group having 1 to 30 carbon atoms, a substituted or unsubstituted aryl group having 6 to 10 carbon atoms, a substituted or unsubstituted alkoxy group having 1 to 30 carbon atoms, a substituted or unsubstituted aryloxy group having 6 to 30 carbon atoms, a substituted or unsubstituted alkoxycarbonyl group having 2 to 30 carbon atoms, a substituted or unsubstituted aryloxycarbonyl group having 7 to 11 carbon atoms, a substituted or unsubstituted carbamoyl group having 3 to 21 carbon atoms, a substituted or unsubstituted acyl group having 2 to 22 carbon atoms, a halogen atom, a hydroxyl group, a cyano group, or a sulfo group.

The present invention will be described in detail below. The components described below are sometimes explained with reference to representative embodiments of the present invention. However, the present invention should not be construed to be limited to these embodiments. In the present specification, “to” denotes a range including numerical values described before and after it as a minimum value and a maximum value.

### [Compound Represented by Formula (1)]

The ultraviolet absorbent (long-wavelength ultraviolet absorbent) comprising the compound represented by Formula (1) will be described below.

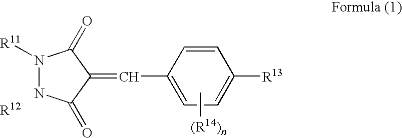

In formula (1), R¹¹, R¹² and R¹⁴ each independently represent a monovalent substituent; R¹³ represents a hydrogen atom or a substituent having a Hammett substituent constant σₚ of −0.35 or more; and n represents an integer of 0 to 4.

The compound represented by the formula (1) is a compound having a skeleton of 4-benzylidenepyrazolidine-3,5-dion. The compound represented by formula (1) is known itself (for example, Bulletin of the Chemical Society of Japan (1932), vol. 7, p. 44). Possibility of application of the compound to various materials such as photographic materials and hologram recording materials is disclosed (see, for example, JP-A-2006-227067 and JP-A-2002-207268).

However, use of the compound as an ultraviolet absorbent has not been known yet. So, it is unexpected that the compound represented by formula (1) has an especially excellent property as a long-wavelength ultraviolet absorbing material.

In formula (1), R¹¹, R¹² and R¹⁴ each independently represent a monovalent substituent.

The monovalent substituent is not specifically defined. Examples thereof include a halogen atom, an aliphatic group [a saturated aliphatic group (this term includes an alkyl group, and a cyclic saturated aliphatic group including a cycloalkyl group, a bicycloalkyl group, a crosslinked cyclic saturated hydrocarbon group, and a spiro-saturated hydrocarbon group), an unsaturated aliphatic group (this term includes a linear unsaturated aliphatic group having a double bond or a triple bond, such as an alkenyl group, an alkynyl group; and a cyclic unsaturated aliphatic group including a cycloalkenyl group, a bicycloalkenyl group, a crosslinked cyclic unsaturated hydrocarbon group, and a spiro-unsaturated hydrocarbon group)], an aryl group (preferably a substituted or unsubstituted phenyl group), a heterocyclic group (preferably a 5- to 8-membered, alicyclic, aromatic or heterocyclic ring having an oxygen atom, a sulfur atom or a nitrogen atom as the ring-constitutive atom, and it may be condensed with a ring such as an aliphatic ring, an aromatic ring and a heterocyclic ring), a cyano group, an aliphatic oxy group (typically an alkoxy group), an aryloxy group, an acyloxy group, a carbamoyloxy group, an aliphatic oxycarbonyloxy group (typically an alkoxycarbonyloxy group), an aryloxycarbonyloxy group, an amino group [including an aliphatic amino group (typically an alkylamino group), an anilino group, and a heterocyclic amino group], an acylamino group, an aminocarbonylamino group, an aliphatic oxycarbonylamino group (typically an alkoxycarbonylamino group), an aryloxycarbonylamino group, a sulfamoylamino group, an aliphatic (typically an alkyl) or aryl sulfonylamino group, an aliphatic thio group (typically an alkylthio group), an arylthio group, a sulfamoyl group, an aliphatic (typically an alkyl) or aryl-sulfinyl group, an aliphatic (typically an alkyl) or aryl-sulfonyl group, an acyl group, an aryloxycarbonyl group, an aliphatic oxycarbonyl group (typically an alkoxycarbonyl group), a carbamoyl group, an aryl or heterocyclic azo group, an imide group, an aliphatic oxysulfonyl group (typically an alkoxysulfonyl group), an aryloxysulfonyl group, a hydroxyl group, a nitro group, a carboxyl group, and a sulfo group. These groups may be further substituted with a substituent (for example, with the substituent mentioned in the above).

In formula (1), R¹³ represents a hydrogen atom or a substituent having a Hammett substituent constant σₚ of −0.35 or more.

The expression “Hammett substituent constant σₚ value” used herein will be briefly described. Hammett's rule is a rule of thumb advocated by L. P. Hammett in 1935 for quantitatively considering the effect of substituents on the reaction or equilibrium of benzene derivatives, and the appropriateness thereof is now widely recognized. The substituent constant determined in the Hammett's rule involves σₚ value and σₘ value. These values can be found in a multiplicity of general publications, and are detailed in, for example, “Lange's Handbook of Chemistry” 12th edition by J. A. Dean, 1979 (McGraw-Hill), “Kagaku no Ryoiki” special issue, No. 122, pp. 96 to 103, 1979 (Nankodo) and Chem. Rev., vol. 91, pp. 165 to 195, 1991.

Examples of the substituent having a Hammett substituent constant σₚ of −0.35 or more include a cyano group (0.66), a carboxyl group (—COOH: 0.45), an alkoxycarbonyl group (e.g. —COOMe: 0.45), an aryloxycarbonyl group (e.g. —COOPh: 0.44), a carbamoyl group (—CONH₂: 0.36), an alkylcarbonyl group (e.g. —COMe: 0.50), an arylcarbonyl group (e.g. —COPh: 0.43), an alkylsulfonyl group (e.g. —SO₂Me: 0.72), an arylsulfonyl group (e.g. —SO₂Ph: 0.68), an acyloxy group (e.g. —OCOMe: 0.31), an alkylsulfonyloxy group (e.g. —OSO₂Me: 0.36), a chlorine atom (0.23), a fluorine atom (0.06), an alkyl group (e.g. —Me: −0.17), an alkoxy group (e.g. —OMe: −0.27), an aryl group (e.g. —C₆H₅: −0.03), an aryloxy group (e.g. —OC₆H₅: −0.03), a sulfo group (e.g. —SO₃H: 0.09) and the like. In the present description, Me represents a methyl group and Ph represents a phenyl group. The values in parenthesis are the σₚ values of typical substituents, as extracted from Chem. Rev., 1991, vol. 91, p. 165 to 195.

In formula (1), n represents an integer of 0 to 4. n is preferably an integer of 0 to 2; more preferably an integer of 0 or 1; and particularly preferably 0.

Here, the substituents represented by R¹¹, R¹², R¹³ and R¹⁴ and substituents on the substituents represented by R¹¹, R¹², R¹³ and R¹⁴ will be more specifically described.

The halogen atom of and on R¹¹, R¹², R¹³ and R¹⁴ includes a fluorine atom, a chlorine atom, a bromine atom, and an iodine atom. Of these, a chlorine atom and a bromine atom are preferable, a chlorine atom is particularly preferable.

The aliphatic group of and on R¹¹, R¹², R¹³ and R¹⁴ includes a linear, branched and cyclic aliphatic groups. The term “saturated aliphatic group” includes an alkyl group, a cycloalkyl group, and a bicycloalkyl group; and these groups may have a substituent. The carbon numbers of these substituents is preferably from 1 to 30. Examples of the alkyl group include a methyl group, an ethyl group, an n-propyl group, an isopropyl group, a t-butyl group, an n-octyl group, an eicosyl group, a 2-chloroethyl group, a 2-cyanoethyl group, a benzyl group and a 2-ethylhexyl group. The cycloalkyl group includes a substituted or unsubstituted cycloalkyl group. The substituted or unsubstituted cycloalkyl group is preferably a cycloalkyl group having 3 to 30 carbon atoms. Examples of the cycloalkyl group include a cyclohexyl group, a cyclopentyl group and a 4-n-dodecylcyclohexyl group. The bicycloalkyl group is preferably a substituted or unsubstituted bicycloalkyl group having 5 to 30 carbon atoms, i.e., a monovalent group obtained by removing one hydrogen atom from a bicycloalkane having 5 to 30 carbon atoms. Examples of the bicycloalkyl group include a bicyclo[1,2,2]heptan-2-yl group and a bicyclo[2,2,2]octan-3-yl group, and a tricyclo or higher structure having three or more ring structures.

The unsaturated aliphatic group of and on R¹¹, R¹², R¹³ and R¹⁴ includes a linear, branched, and cyclic unsaturated aliphatic groups. The unsaturated aliphatic group includes an alkenyl group, a cycloalkenyl group, a bicycloalkenyl group and an alkynyl group. The alkenyl group includes a linear, branched, and cyclic substituted or unsubstituted alkenyl groups. The alkenyl group is preferably a substituted or unsubstituted alkenyl group having 2 to 30 carbon atoms. Examples of the alkenyl group include a vinyl group, an allyl group, a prenyl group, a geranyl group, and an oleyl group. The cycloalkenyl group is preferably a substituted or unsubstituted cycloalkenyl group having 3 to 30 carbon atoms, i.e., a monovalent group obtained by removing one hydrogen atom from a cycloalkene having 3 to 30 carbon atoms. Examples of the cycloalkenyl group include a 2-cyclopenten-1-yl group and a 2-cyclohexen-1-yl group. The bicycloalkenyl group includes a substituted or unsubstituted bicycloalkenyl group, and preferably a substituted or unsubstituted bicycloalkenyl group having 5 to 30 carbon atoms, i.e., a monovalent group obtained by removing one hydrogen atom from a bicycloalkene having one double bond. Examples of the bicycloalkenyl group include a bicyclo[2,2,1]hept-2-en-1-yl group and a bicyclo[2,2,2]oct-2-en-4-yl group. The alkynyl group is preferably a substituted or unsubstituted alkynyl group having 2 to 30 carbon atoms, e.g., an ethynyl group, or a propargyl group.

The aryl group of or on R¹¹, R¹², R¹³ and R¹⁴ is preferably a substituted or unsubstituted aryl group having 6 to 30 carbon atoms, e.g., a phenyl group, a p-tolyl group, a naphthyl group, an m-chlorophenyl group, or an o-hexadecanoylaminophenyl group. The aryl group is more preferably a phenyl group which may have a substituent.

The heterocyclic group of or on R¹¹, R¹², R¹³ and R¹⁴ is a monovalent group obtained by removing one hydrogen atom from a substituted or unsubstituted, aromatic or non-aromatic heterocyclic compound, which may be condensed to another ring. The heterocyclic group is preferably a 5- or 6-membered heterocyclic group. The hetero atom(s) constituting the heterocyclic group is preferably an oxygen atom, a sulfur atom, or a nitrogen atom. The heterocyclic group is more preferably a 5- or 6-membered aromatic heterocyclic group having 3 to 30 carbon atoms. The hetero ring in the heterocyclic group are exemplified below: a pyridine ring, a pyrazine ring, a pyridazine ring, a pyrimidine ring, a triazine ring, a quinoline ring, an isoquinoline ring, a quinazoline ring, a cinnoline ring, a phthalazine ring, a quinoxaline ring, a pyrrole ring, an indole ring, a furan ring, a benzofuran ring, a thiophene ring, a benzothiophene ring, a pyrazole ring, an imidazole ring, a benzimidazole ring, a triazole ring, an oxazole ring, a benzoxazole ring, a thiazole ring, a benzothiazole ring, an isothiazole ring, a benzisothiazole ring, a thiadiazole ring, an isoxazole ring, a benzisoxazole ring, a pyrrolidine ring, a piperidine ring, a piperazine ring, an imidazolidine ring and a thiazoline ring.

The aliphatic oxy group (as a representative example, an alkoxy group) of or on R¹¹, R¹², R¹³ and R¹⁴ includes a substituted or unsubstituted aliphatic oxy group (as a representative example, alkoxy group). The substituted or unsubstituted aliphatic oxy group is preferably an aliphatic oxy group having 1 to 30 carbon atoms, e.g., a methoxy group, an ethoxy group, an isopropoxy group, an n-octyloxy group, a methoxyethoxy group, a hydroxyethoxy group, or a 3-carboxypropoxy group.

The aryloxy group of or on R¹¹, R¹², R¹³ and R¹⁴ is preferably a substituted or unsubstituted aryloxy group having 6 to 30 carbon atoms, e.g., a phenoxy group, a 2-methylphenoxy group, a 4-t-butylphenoxy group, a 3-nitrophenoxy group, or a 2-tetradecanoylaminophenoxy group. The aryloxy group is more preferably a phenoxy group which may have a substituent.

The acyloxy group of or on R¹¹, R¹², R¹³ and R¹⁴ is preferably a formyloxy group, a substituted or unsubstituted alkylcarbonyloxy group having 2 to 30 carbon atoms, or a substituted or unsubstituted arylcarbonyloxy group having 6 to 30 carbon atoms, e.g., a formyloxy group, an acetyloxy group, a pivaloyloxy group, a stearoyloxy group, a benzoyloxy group, or a p-methoxyphenylcarbonyloxy group.

The carbamoyloxy group of or on R¹¹, R¹², R¹³ and R¹⁴ is preferably a substituted or unsubstituted carbamoyloxy group having 1 to 30 carbon atoms, e.g., an N,N-dimethylcarbamoyloxy group, an N,N-diethylcarbamoyloxy group, a morpholinocarbonyloxy group, an N,N-di-n-octylaminocarbonyloxy group, or an N-n-octylcarbamoyloxy group.

The aliphatic oxy carbonyloxy group (as a representative example, an alkoxycarbonyloxy group) of or on R¹¹, R¹², R¹³ and R¹⁴ is preferably an aliphatic oxy carbonyloxy group having 2 to 30 carbon atoms. The aliphatic oxy carbonyloxy group may have a substituent. There can be exemplified a methoxycarbonyloxy group, an ethoxycarbonyloxy group, a t-butoxycarbonyloxy group, or an n-octylcarbonyloxy group.

The aryloxycarbonyloxy group of or on R¹¹, R¹², R¹³ and R¹⁴ is preferably a substituted or unsubstituted aryloxycarbonyloxy group having 7 to 30 carbon atoms, e.g., a phenoxycarbonyloxy group, a p-methoxyphenoxycarbonyloxy group, or a p-n-hexadecyloxyphenoxycarbonyloxy group. The aryloxycarbonyloxy group is more preferably a phenoxycarbonyloxy group which may have a substituent.

The amino group of or on R¹¹, R¹², R¹³ and R¹⁴ includes an unsubstituted amino group, an aliphatic amino group (as a representative example, an alkylamino group), an arylamino group, and a heterocyclic amino group. The amino group is preferably a substituted or unsubstituted aliphatic amino group (as a representative example, alkylamino group) having 1 to 30 carbon atoms, or a substituted or unsubstituted arylamino group having 6 to 30 carbon atoms, e.g., an amino group, a methylamino group, a dimethylamino group, an anilino group, an N-methyl-anilino group, a diphenylamino group, a hydroxyethylamino group, a carboxyethylamino group, a sulfoethylamino group, a 3,5-dicarboxyanilino group, or a 4-quinolylamino group.

The acylamino group of or on R¹¹, R¹², R¹³ and R¹⁴ is preferably a formylamino group, a substituted or unsubstituted alkylcarbonylamino group having 1 to 30 carbon atoms, or a substituted or unsubstituted arylcarbonylamino group having 6 to 30 carbon atoms, e.g., a formylamino group, an acetylamino group, a pivaloylamino group, a lauroylamino group, a benzoylamino group, or a 3,4,5-tri-n-octyloxyphenylcarbonylamino group.

The aminocarbonylamino group of or on R¹¹, R¹², R¹³ and R¹⁴ is preferably a substituted or unsubstituted aminocarbonylamino group having 1 to 30 carbon atoms, e.g., a carbamoylamino group, an N,N-dimethylaminocarbonylamino group, an N,N-diethylaminocarbonylamino group, or a morpholinocarbonylamino group. In the aminocarbonylamino group, the term “amino” has the same meaning as “amino” in the above-described amino group.

The aliphatic oxy carbonylamino group (as a representative example, alkoxycarbonylamino group) of or on R¹¹, R¹², R¹³ and R¹⁴ is preferably a substituted or unsubstituted aliphatic oxy carbonylamino group having 2 to 30 carbon atoms, e.g., a methoxycarbonylamino group, an ethoxycarbonylamino group, a t-butoxycarbonylamino group, an n-octadecyloxycarbonylamino group, or an N-methyl-methoxycarbonylamino group.

The aryloxycarbonylamino group of or on R¹¹, R¹², R¹³ and R¹⁴ is preferably a substituted or unsubstituted aryloxycarbonylamino group having 7 to 30 carbon atoms, e.g., a phenoxycarbonylamino group, a p-chlorophenoxycarbonylamino group, or an m-(n-octyloxy)phenoxycarbonylamino group. The aryloxycarbonylamino group is more preferably a phenoxycarbonylamino group which may have a substituent.

The sulfamoylamino group of or on R¹¹, R¹², R¹³ and R¹⁴ is preferably a substituted or unsubstituted sulfamoylamino group having 0 to 30 carbon atoms, e.g., a sulfamoylamino group, an N,N-dimethylaminosulfonylamino group, or an N-n-octylaminosulfonylamino group.

The aliphatic- (as a representative example, alkyl-) or aryl-sulfonylamino group of or on R¹¹, R¹², R¹³ and R¹⁴ is preferably a substituted or unsubstituted aliphatic sulfonylamino group (as a representative example, alkylsulfonylamino group) having 1 to 30 carbon atoms, or a substituted or unsubstituted arylsulfonylamino group (preferably a phenylsulfonylamino group which may have a substituent(s)) having 6 to 30 carbon atoms, e.g., a methylsulfonylamino group, a butylsulfonylamino group, a phenylsulfonylamino group, a 2,3,5-trichlorophenylsulfonylamino group, or a p-methylphenylsulfonylamino group.

The aliphatic thio group (as a representative example, alkylthio group) of or on R¹¹, R¹², R¹³ and R¹⁴ is preferably a substituted or unsubstituted alkylthio group having 1 to 30 carbon atoms, e.g., a methylthio group, an ethylthio group, or an n-hexadecylthio group.

The aryl thio group of or on R¹¹, R¹², R¹³ and R¹⁴ is preferably a substituted or unsubstituted aryl thio group having 6 to 12 carbon atoms, e.g., a phenylthio group, a 1-naphthylthio group, or a 2-naphthylthio group.

The sulfamoyl group of or on R¹¹, R¹², R¹³ and R¹⁴ is preferably a substituted or unsubstituted sulfamoyl group having 0 to 30 carbon atoms, e.g., an N-ethylsulfamoyl group, an N-(3-dodecyloxypropyl)sulfamoyl group, an N,N-dimethylsulfamoyl group, an N-acetylsulfamoyl group, an N-benzoylsulfamoly group, or an N-(N′-phenylcarbamoyl)sulfamoyl group.

The aliphatic- (as a representative example, alkyl-) or aryl-sulfinyl group of or on R¹¹, R¹², R¹³ and R¹⁴ is preferably a substituted or unsubstituted aliphatic sulfinyl group (as a representative example, alkylsulfinyl group) having 1 to 30 carbon atoms, or a substituted or unsubstituted arylsulfinyl group (preferably a phenylsulfinyl group which may have a substituent(s)) having 6 to 30 carbon atoms, e.g., a methylsulfinyl group, an ethylsulfinyl group, a phenylsulfinyl group, or a p-methylphenylsulfinyl group.

The aliphatic- (as a representative example, alkyl-) or aryl-sulfonyl group of or on R¹¹, R¹², R¹³ and R¹⁴ is preferably a substituted or unsubstituted aliphatic-sulfonyl group (as a representative example, alkylsulfonyl group) having 1 to 30 carbon atoms, or a substituted or unsubstituted arylsulfonyl group (preferably a phenylsulfonyl group which may have a substituent(s)) having 6 to 30 carbon atoms, e.g., a methylsulfonyl group, an ethylsulfonyl group, a phenylsulfonyl group, or a p-toluenesulfonyl group.

The acyl group of or on R¹¹, R¹², R¹³ and R¹⁴ is preferably a formyl group, a substituted or unsubstituted aliphatic carbonyl group (as a representative example, alkylcarbonyl group) having 2 to 30 carbon atoms, a substituted or unsubstituted arylcarbonyl group (preferably a phenylcarbonyl group which may have a substituent(s)) having 7 to 30 carbon atoms, or a substituted or unsubstituted heterocyclic carbonyl group having 4 to 30 carbon atoms and being bonded to said carbonyl group through a carbon atom, e.g., an acetyl group, a pivaloyl group, a 2-chloroacetyl group, a stearoyl group, a benzoyl group, a p-n-octyloxyphenylcarbonyl group, a 2-pyridylcarbonyl group, or a 2-furylcarbonyl group.

The aryloxycarbonyl group of or on R¹¹, R¹², R¹³ and R¹⁴ is preferably a substituted or unsubstituted aryloxycarbonyl group having 7 to 30 carbon atoms, e.g., a phenoxycarbonyl group, an o-chlorophenoxycarbonyl group, an m-nitrophenoxycarbonyl group, or a p-(t-butyl)phenoxycarbonyl group. The aryloxycarbonyl group is more preferably a phenoxycarbonyl group which may have a substituent.

The aliphatic oxycarbonyl group (as a representative example, alkoxycarbonyl group) of or on R¹¹, R¹², R¹³ and R¹⁴ is preferably a substituted or unsubstituted aliphatic oxycarbonyl group having 2 to 30 carbon atoms, e.g., a methoxycarbonyl group, an ethoxycarbonyl group, a t-butoxycarbonyl group, or an n-octadecyloxycarbonyl group.

The carbamoyl group of or on R¹¹, R¹², R¹³ and R¹⁴ is preferably a substituted or unsubstituted carbamoyl group having 1 to 30 carbon atoms, e.g., a carbamoyl group, an N-methylcarbamoyl group, an N,N-dimethylcarbamoyl group, an N,N-di-n-octylcarbamoyl group, or an N-(methylsulfonyl)carbamoyl group.

Examples of the aryl- or heterocyclic-azo group of or on R¹¹, R¹², R¹³ and R¹⁴ include a phenylazo group, a 4-methoxyphenylazo group, a 4-pivaloylaminophenylazo group, and a 2-hydroxy-4-propanoylphenylazo group.

Examples of the imido group of or on R¹¹, R¹², R¹³ and R¹⁴ include an N-succinimido group and an N-phthalimido group.

The aliphatic oxysulfonyl group of or on R¹¹, R¹², R¹³ and R¹⁴ is preferably an aliphatic oxysulfonyl group having 1 to 30 carbon atoms, e.g., a methoxysulfonyl group, an ethoxysulfonyl group, and a n-butoxysulfonyl group.

The aryloxysulfonyl group of or on R¹¹, R¹², R¹³ and R¹⁴ is preferably a substituted or unsubstituted aryloxysulfonyl group having 6 to 12 carbon atoms, e.g., a phenoxysulfonyl group and a 2-naphthoxyphenyl group.

In addition to these substituents, examples of the substituent of or on R¹¹, R¹², R¹³and R¹⁴ include a hydroxyl group, a cyano group, a nitro group, a sulfo group, a carboxyl group, and the like.

These groups may each further have a substituent. Examples of the substituent include the above-mentioned substituents.

In formula (1), R¹¹ and R¹² each independently are preferably a substituted or unsubstituted alkyl group having 1 to 30 carbon atoms, an allyl group, a substituted or unsubstituted aryl group having 6 to 20 carbon atoms, a substituted or unsubstituted heterocyclic group, a substituted or unsubstituted acyl group having 2 to 30 carbon atoms, a substituted or unsubstituted alkoxycarbonyl group having 2 to 30 carbon atoms, a substituted or unsubstituted aryloxycarbonyloxy group having 7 to 30 carbon atoms, a substituted or unsubstituted carbamoyl group, a substituted or unsubstituted alkylsulfonyl group having 1 to 30 carbon atoms, or a substituted or unsubstituted arylsulfonyl group having 6 to 30 carbon atoms; more preferably a substituted or unsubstituted alkyl group having 1 to 20 carbon atoms, a substituted or unsubstituted aryl group having 6 to 20 carbon atoms, a substituted or unsubstituted acyl group having 2 to 20 carbon atoms, or a substituted or unsubstituted alkoxycarbonyl group having 2 to 20 carbon atoms; and particularly preferably a substituted or unsubstituted alkyl group having 1 to 18 carbon atoms.

In formula (1), R¹³ is preferably a hydrogen atom, a substituted or unsubstituted alkyl group having 1 to 30 carbon atoms, a substituted or unsubstituted aryl group having 6 to 10 carbon atoms, a substituted or unsubstituted alkoxy group having 1 to 30 carbon atoms, a substituted or unsubstituted aryloxy group having 6 to 30 carbon atoms, a substituted or unsubstituted alkoxycarbonyl group having 2 to 30 carbon atoms, a substituted or unsubstituted aryloxycarbonyl group having 7 to 11 carbon atoms, a substituted or unsubstituted carbamoyl group having 3 to 21 carbon atoms, a substituted or unsubstituted acyl group having 2 to 22 carbon atoms, a halogen atom, a cyano group, or a sulfo group; more preferably a hydrogen atom, a substituted or unsubstituted alkyl group having 1 to 20 carbon atoms, a substituted or unsubstituted alkoxy group having 1 to 20 carbon atoms, a substituted or unsubstituted alkoxycarbonyl group having 2 to 20 carbon atoms, a substituted or unsubstituted carbamoyl group having 3 to 16 carbon atoms, a fluorine atom, or a chlorine atom; and particularly preferably an unsubstituted alkyl group having 1 to 12 carbon atoms, an unsubstituted alkoxy group having 1 to 12 carbon atoms, an unsubstituted alkoxycarbonyl group having 2 to 10 carbon atoms, or a chlorine atom.

In formula (1), R¹⁴ is preferably a substituted or unsubstituted alkyl group having 1 to 30 carbon atoms, a substituted or unsubstituted aryl group having 6 to 30 carbon atoms, a halogen atom, or a cyano group; more preferably a substituted or unsubstituted alkyl group having 1 to 20 carbon atoms; and particularly preferably an alkyl group having 1 to 8 carbon atoms.

The following is an explanation about a preferable combination of various substituents (atoms) that a compound represented by formula (1) may have (combination of R¹¹, R¹², R¹³ and R¹⁴): A preferred compound is a compound in which at least one of the substituents of R¹¹, R¹², R¹³ and R¹⁴ is the above-described preferable substituent. A more preferred compound is a compound in which two or more substituents of R¹¹, R¹², R¹³ and R¹⁴ are the above-described preferable substituents. The most preferred compound is a compound in which all substituents of R¹¹, R¹², R¹³ and R¹⁴ are the above-described preferable substituents.

Examples of a preferred combination of R¹¹, R¹², R¹³, R¹⁴, and n in the compound represented by formula (1) include combinations wherein R¹¹ and R¹² each independently are a substituted or unsubstituted alkyl group having 1 to 12 carbon atoms, a substituted or unsubstituted phenyl group, or a substituted or unsubstituted alkoxycarbonyl group having 2 to 10 carbon atoms; R¹³ is a hydrogen atom, a substituted or unsubstituted alkyl group having 1 to 12 carbon atoms, or a substituted or unsubstituted alkoxy group having 1 to 12 carbon atoms; R¹⁴ is a chlorine atom, a fluorine atom, or a substituted or unsubstituted alkyl group having 1 to 12 carbon atoms; and n is 0 or 1.

In more preferred combinations thereof, R¹¹ and R¹² each independently are an unsubstituted alkyl group having 1 to 10 carbon atoms, a substituted or unsubstituted phenyl group, or an unsubstituted alkoxycarbonyl group having 2 to 10 carbon atoms; R¹³ is a hydrogen atom, an unsubstituted alkyl group having 1 to 10 carbon atoms, or a substituted or unsubstituted alkoxy group having 1 to 10 carbon atoms; R¹⁴ is a chlorine atom, a fluorine atom, or an unsubstituted alkyl group having 1 to 10 carbon atoms; and n is 0 or 1.

In the most preferred combinations thereof, R¹¹ and R¹² each independently are an unsubstituted alkyl group having 1 to 8 carbon atoms, an unsubstituted phenyl group, or an unsubstituted alkoxycarbonyl group having 2 to 8 carbon atoms; R¹³ is a hydrogen atom, an unsubstituted alkyl group having 1 to 8 carbon atoms, or an unsubstituted alkoxy group having 1 to 8 carbon atoms; and n is 0.

### [Compound Represented by Formula (2)]

The compound represented by formula (1) is preferably a compound represented by formula (2).

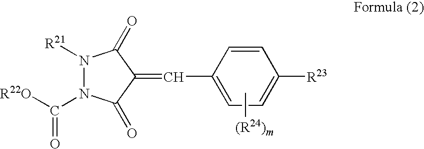

wherein R²¹ represents a substituted or unsubstituted alkyl group having 1 to 18 carbon atoms, or a substituted or unsubstituted phenyl group; R²² represents a substituted or unsubstituted alkyl group having 1 to 18 carbon atoms; R²³ represents a hydrogen atom or a substituent having a Hammett substituent constant σₚ of −0.35 or more; R²⁴ represents a monovalent substituent; and m represents an integer of 0 to 4.

The compound represented by formula (2) is a 4-benzylidenepyrazolidine-3,5-dion compound in which the substituent at 1-position of the pyrazolidine moiety is an alkyl group or a phenyl group (i.e., R²¹ in formula (2)), and the substituent at 2-position of the pyrazolidine moiety is an alkoxycarbonyl group (i.e., —COOR²² in formula (2)). It is unexpected that, among the 4-benzylidenepyrazolidine-3,5-dion compounds, the compound represented by formula (2) has excellent properties such as usefulness as an ultraviolet absorbent as well as less reduction of absorption capacity with the lapse of time. Further, the compound represented by formula (2) has such excellent properties that the density at toe of the long wavelength side end portion remarkably decreases in both absorption spectrum and transmission spectrum; and the compound is a clear and colorless compound having a small absorption at wavelength range of around 450 nm to 550 nm, at which range human luminosity factor (visibility) is especially high. In other words, when the compound represented by formula (2) is incorporated in a polymer material, almost no coloration is caused by the compound, and consequently the polymer material does not turn to yellow.

In formula (2), R²¹ represents a substituted or unsubstituted alkyl group having 1 to 18 carbon atoms, or a substituted or unsubstituted phenyl group. The substituent that R²¹ may have, has the same meaning as the substituent on R¹¹, R¹², R¹³ and R¹⁴ in formula (1), and the preferable range is also the same.

In formula (2), R²² represents a substituted or unsubstituted alkyl group having 1 to 18 carbon atoms. The substituent that R²² may have, has the same meaning as the substituent on R¹¹, R¹², R¹³ and R¹⁴ in formula (1), and the preferable range is also the same.

In formula (2), R²³, R²⁴ and m each have the same meaning as of R¹³, R¹⁴ and n in formula (1), and the preferable ranges are also the same.

The following is an explanation about a preferable combination of various substituents (atoms) that a compound represented by formula (2) may have (combination of R²¹, R²², R²³ and R²⁴): A preferred compound is a compound in which at least one of the substituents of R²¹, R²², R²³ and R²⁴ is the above-described preferable substituent. A more preferred compound is a compound in which two or more substituents of R²¹, R²², R²³ and R²⁴ are the above-described preferable substituents. The most preferred compound is a compound in which all substituents of R²¹, R²², R²³ and R²⁴ are the above-described preferable substituents.

Examples of a preferred combination of R²¹, R²², R²³, R²⁴, and m in the compound represented by formula (2) include combinations wherein R²¹ is a substituted or unsubstituted alkyl group having 1 to 16 carbon atoms, or a substituted or unsubstituted phenyl group; R²² is a substituted or unsubstituted alkyl group having 1 to 16 carbon atoms; R²³ is a hydrogen atom, a substituted or unsubstituted alkyl group having 1 to 12 carbon atoms, or a substituted or unsubstituted alkoxy group having 1 to 12 carbon atoms; R²⁴ is a chlorine atom, a fluorine atom, or a substituted or unsubstituted alkyl group having 1 to 12 carbon atoms; and m is 0 or 1.

In more preferred combinations thereof, R²¹ is a substituted or unsubstituted alkyl group having 1 to 12 carbon atoms, or a mono-substituted or unsubstituted phenyl group; R²² is a substituted or unsubstituted alkyl group having 1 to 12 carbon atoms; R²³ is a hydrogen atom, an unsubstituted alkyl group having 1 to 10 carbon atoms, or a substituted or unsubstituted alkoxy group having 1 to 10 carbon atoms; R²⁴ is a chlorine atom, a fluorine atom, or an unsubstituted alkyl group having 1 to 10 carbon atoms; and m is 0 or 1.

In the most preferred combinations thereof, R²¹ is an unsubstituted alkyl group having 1 to 8 carbon atoms, or an unsubstituted phenyl group; R²² is an unsubstituted alkyl group having 1 to 8 carbon atoms; R²³ is a hydrogen atom, an unsubstituted alkyl group having 1 to 8 carbon atoms, or an unsubstituted alkoxy group having 1 to 8 carbon atoms; and m is 0.

A molecular weight of the compound represented by formula (1) is preferably 1500 or less, more preferably 1000 or less, and further more preferably from 400 to 900, from viewpoints of both ultraviolet absorption capacity and resistance to bleeding.

Specific examples of the compound represented by (1) are shown in the followings, but the present invention is not limited thereto.

TABLE 1

| | | | | | | Sub- |
|---|---|---|---|---|---|---|
| | | | | | | stitution |
| | | | | | | position |
| | | | | | | of R¹⁴ |
| | | | | | | (to the |
| | | | | | | methine |
| No. | R¹¹ | R¹² | R¹³ | R¹⁴ | n | group) |
| 1 | Methyl | Methyl | Methoxy | — | 0 | |
| 2 | Phenyl | Phenyl | Methyl | — | 0 | |
| 3 | Phenyl | Phenyl | Methoxy | — | 0 | |
| 4 | Methyl | Methyl | Hydrogen atom | Methoxy | 1 | o-Position |
| 5 | Methyl | Methyl | Hydrogen atom | Methoxy | 1 | m-Position |
| 6 | Methyl | Methyl | t-Butyl | — | 0 | |
| 7 | Methyl | Methyl | Methoxy | — | 0 | |
| | | | carbonyl | | | |
| 8 | Methyl | Methyl | Acetoxy | — | 0 | |
| 9 | Methyl | Methyl | Hydrogen atom | Hydroxyl | 1 | o-Position |
| 10 | Methyl | Methyl | Hydrogen atom | — | 0 | |
| 11 | Phenyl | Phenyl | Ethyl | — | 0 | |
| 12 | Phenyl | Phenyl | iso-Propyl | — | 0 | |
| 13 | Phenyl | Phenyl | t-Butyl | — | 0 | |
| 14 | Phenyl | Phenyl | Hydrogen atom | — | 0 | |
| 15 | Phenyl | Phenyl | Hydrogen atom | Chlorine | 1 | o-Position |
| | | | | atom | | |
| 16 | Phenyl | Phenyl | Hydrogen atom | Methyl | 1 | o-Position |
| 17 | Phenyl | Phenyl | Acetoxy | — | 0 | |
| 18 | Phenyl | Phenyl | Methoxy | — | 0 | |
| | | | carbonyl | | | |
| 19 | Ethoxy | Ethoxy | Methoxy | — | 0 | |
| | car- | carbonyl | | | | |
| | bonyl | | | | | |
| 20 | Ethoxy | Ethoxy | Hydrogen atom | — | 0 | |
| | car- | carbonyl | | | | |
| | bonyl | | | | | |
| 21 | Ethoxy | Ethoxy | Methyl | — | 0 | |
| | car- | carbonyl | | | | |
| | bonyl | | | | | |
| 22 | Phenyl | Ethoxy | Methoxy | — | 0 | |
| | | carbonyl | | | | |
| 23 | Phenyl | Ethoxy | Methyl | — | 0 | |
| | | carbonyl | | | | |
| 24 | Phenyl | Ethoxy | Methoxy | Methoxy | 1 | o-Position |
| | | carbonyl | | | | |
| 25 | Phenyl | Ethoxy | Ethoxy | — | 0 | |
| | | carbonyl | | | | |
| 26 | Phenyl | Ethoxy | Chlorine atom | — | 0 | |
| | | carbonyl | | | | |
| 27 | Phenyl | Ethoxy | Hydrogen atom | — | 0 | |
| | | carbonyl | | | | |
| 28 | Phenyl | Ethoxy | Methoxy | — | 0 | |
| | | carbonyl | carbonyl | | | |
| 29 | t-Butyl | Ethoxy | Methyl | — | 0 | |
| | | carbonyl | | | | |
| 30 | t-Butyl | Ethoxy | Methoxy | — | 0 | |
| | | carbonyl | | | | |
| 31 | Phenyl | Acetyl | Hydrogen atom | — | 0 | |
| 32 | Phenyl | Acetyl | Methoxy | — | 0 | |

The exemplified compounds (22) to (30) are included in not only the compound represented by formula (1) but also the compound represented by formula (2).

These compounds may be synthesized in accordance with known synthetic methods of similar compounds. For example, these compounds can be synthesized by referring to the method described in Bull. Chem. Soc. Jpn., 1932, vol. 7, p. 45 to 48.

Specifically, for example, object compounds may be obtained by reacting a benzaldehyde derivative represented by formula (3) and a pyrazolidinedion compound represented by formula (4). More specifically, details will be described in Examples.

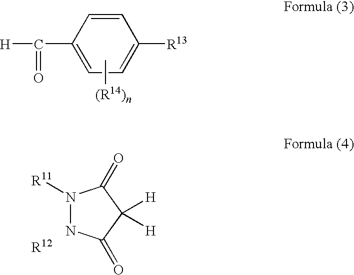

In formula (3), R¹³, R¹⁴ and n each have the same meaning as of R¹³, R¹⁴ and n in formula (1), and the preferable ranges are also the same.

In formula (4), R¹¹ and R¹² each have the same meaning as of R¹¹ and R¹² in formula (1), and the preferable ranges are also the same.

The compound represented by formula (3) is available as a marketed product (for example, catalog No. 019-04133, manufactured by Waco Pure Chemical Industries, Co., Ltd.).

The compound represented by formula (4) can be synthesized by a method described in, for example, Monatsh. Chem., 112 (1981), p. 369. Alternatively, the compound is available as a marketed product (for example, catalog No. 322-39723, manufactured by Waco Pure Chemical Industries, Co., Ltd.).

The compound represented by formula (1) is preferably used as a long-wavelength ultraviolet absorbent. The maximum absorption wavelength of the compound is preferably in the range of 300 nm to 420 nm, more preferably from 330 nm to 410 nm, and furthermore preferably from 360 nm to 400 nm.

The ultraviolet absorbent is used in preparation of the polymer material according to the present invention. The polymer material of the present invention contains a polymer substance described below and the ultraviolet absorbent comprising the compound represented by formula (1) or (2).

The ultraviolet absorbent comprising the compound represented by formula (1) or (2) is contained in the polymer substance in various methods. When the ultraviolet absorbent comprising the compound represented by formula (1) or (2) is compatible with the polymer substance, the ultraviolet absorbent comprising the compound represented by formula (1) or (2) may be added to the polymer substance directly. The ultraviolet absorbent comprising the compound represented by formula (1) or (2) may be dissolved in a cosolvent compatible with the polymer substance, and then the obtained solution be added to the polymer substance. The ultraviolet absorbent comprising the compound represented by formula (1) or (2) may be dispersed in a polymer, and the obtained dispersion be added to the polymer substance.

The method of adding the ultraviolet absorbent comprising the compound represented by formula (1) or (2) to the polymer substance is determined, by reference to the description in JP-A-58-209735, JP-A-63-264748, JP-A-4-191851, JP-A-8-272058, and British Patent No. 2016017A.

In the present invention, an ultraviolet absorbent comprising two or more kinds of compounds represented by formula (1) or (2) different in chemical structure may be used in combination. Alternatively, the compound in the present invention and one or more kinds of ultraviolet absorbents different in chemical structure may be used in combination. Two kinds (preferably three kinds) of ultraviolet absorbents when used in combination absorb ultraviolet ray in a wider wavelength range. In addition, the use of two or more kinds of ultraviolet absorbents in combination has a function to stabilize the dispersion state.

Any ultraviolet absorbent having a chemical structure other than that of ultraviolet absorbent in the present invention may be used. Examples thereof include those described, for example, in Yasuichi Okatsu Ed., “Development of Polymer Additives and Environmental Measures” (CMC Publishing, 2003), Chapter 2; and Toray Research Center Inc., Technical Survey Dept., Ed., “New Trend of Functional Polymer Additives” (Toray Research Center Inc., 1999), Chapter 2.3.1. Examples thereof include ultraviolet absorbing structures such as triazine-based, benzotriazole-based, benzophenone-based, merocyanine-based, cyanine-based, dibenzoylmethane-based, cinnamic acid-based, acrylate-based, benzoic ester-based, and oxalic diamide-based compounds. Specific examples thereof are described, for example, in Fine Chemicals, 2004, May, p. 28 to 38; Toray Research Center Inc., Technical Survey Dept., Ed., “New Trend of Functional Polymer Additives” (Toray Research Center Inc., 1999), p. 96 to 140; and Yasuichi Okatsu Ed., “Development of Polymer Additives and Environmental Measures” (CMC Publishing, 2003), p. 54 to 64.

Among these, preferable are benzotriazole-based, benzophenone-based, salicylic acid-based, acrylate-based, and triazine-based compounds. More preferable are benzotriazole-based, benzophenone-based, and triazine-based compounds. Particularly preferable are benzotriazole-based and triazine-based compounds.

The effective absorption wavelength of benzotriazole-based compounds is approximately 270 to 380 nm, and specific examples thereof include 2-(2′-hydroxy-5′-methylphenyl)benzotriazole, 2-(2′-hydroxy-5′-t-butylphenyl)benzotriazole, 2-(2′-hydroxy-3′-t-butyl-5′-methylphenyl)-5-chlorobenzotriazole, 2-(2′-hydroxy-3′,5′-di-t-butylphenyl)-5-chlorobenzotriazole, 2-(2′-hydroxy-3′-dodecyl-5′-methylphenyl)-5-chlorobenzotriazole, 2-(2′-hydroxy-3′,5′-di-t-amylphenyl)benzotriazole, 2-(2′-hydroxy-3′,5′-di-t-amylphenyl)benzotriazole, 2-(2′-hydroxy-5′-(1,1,3,3-tetramethylbutyl)phenyl)benzotriazole, 2-(2′-hydroxy-4′-octyloxyphenyl)benzotriazole, 2-(2′-hydroxy-3′-(3,4,5,6-tetrahydrophthalimidylmethyl)-5′-methylbenzyl)phenyl)benzotriazole, 2-(3′-sec-butyl-5′-t-butyl-2′-hydroxyphenyl)benzotriazole, 2-(3′,5′-bis-(α,α-dimethylbenzyl)-2′-hydroxyphenyl)benzotriazole, 2-(3′-t-butyl-2′-hydroxy-5′-(2-octyloxycarbonylethyl)phenyl)-5-chloro-benzotriazole, 2-(3′-t-butyl-5′-[2-(2-ethylhexyloxy)-carbonylethyl]-2′-hydroxyphenyl)-5-chloro-benzotriazole, 2-(3′-t-butyl-2′-hydroxy-5′-(2-methoxycarbonylethyl)phenyl)-5-chloro-benzotriazole, 2-(3′-t-butyl-2′-hydroxy-5′-(2-methoxycarbonylethyl)phenyl)benzotriazole, 2-(3′-t-butyl-2′-hydroxy-5′-(2-octyloxycarbonylethyl)phenyl)benzotriazole, 2-(3′-t-butyl-5′-[2-(2-ethylhexyloxy)carbonylethyl]-2′-hydroxyphenyl)benzotriazole, 2-(3′-dodecyl-2′-hydroxy-5′-methylphenyl)benzotriazole, 2-(3′-t-butyl-2′-hydroxy-5′-(2-isooctyloxycarbonylethyl)phenylbenzotriazole, 2,2′-methylene-bis[4-(1,1,3,3-tetramethylbutyl)-6-benzotriazole-2-ylphenol], and the like.

The effective absorption wavelength of triazine-based compounds is approximately 270 to 380 nm, and specific examples thereof 2-(4-butoxy-2-hydroxyphenyl)-4,6-di(4-butoxyphenyl)-1,3,5-triazine, 2-(4-butoxy-2-hydroxyphenyl)-4,6-di(2,4-dibutoxyphenyl)-1,3,5-triazine, 2,4-di(4-butoxy-2-hydroxyphenyl)-6-(4-butoxyphenyl)-1,3,5-triazine, 2,4-di(4-butoxy-2-hydroxyphenyl)-6-(2,4-dibutoxyphenyl)-1,3,5-triazine, 2,4,6-tris(2-hydroxy-4-octyloxyphenyl)-1,3,5-triazine, 2-(2-hydroxy-4-octyloxyphenyl)-4,6-bis(2,4-dimethylphenyl)-1,3,5-triazine, 2-(2,4-dihydroxyphenyl)-4,6-bis(2,4-dimethylphenyl)-1,3,5-triazine, 2,4-bis(2-hydroxy-4-propyloxyphenyl)-6-(2,4-dimethylphenyl)-1,3,5-triazine, 2-(2-hydroxy-4-octyloxyphenyl)-4,6-bis(4-methylphenyl)-1,3,5-triazine, 2-(2-hydroxy-4-dodecyloxyphenyl)-4,6-bis(2,4-dimethylphenyl)-1,3,5-triazine, 2-(2-hydroxy-4-tridecyloxyphenyl)-4,6-bis(2,4-dimethylphenyl)-1,3,5-triazine, 2-[2-hydroxy-4-(2-hydroxy-3 -butyloxypropoxy)phenyl]-4,6-bis(2,4-dimethyl)-1,3,5-triazine, 2-[2-hydroxy-4-(2-hydroxy-3-octyloxypropyloxy)phenyl]-4,6-bis(2,4-dimethyl)-1,3,5-triazine, 2-[4-(dodecyloxy/tridecyloxy-2-hydroxypropoxy)-2-hydroxyphenyl]-4,6-bis(2,4-dimethylphenyl)-1,3,5-triazine, 2-[2-hydroxy-4-(2-hydroxy-3-dodecyloxypropoxy)phenyl]-4,6-bis(2,4-dimethylphenyl)-1,3,5-triazine, 2-(2-hydroxy-4-hexyloxy)phenyl-4,6-diphenyl-1,3,5-triazine, 2-(2-hydroxy-4-methoxyphenyl)-4,6-diphenyl-1,3,5-triazine, 2,4,6-tris(2-hydroxy-4-(3-butoxy-2-hydroxypropoxy)phenyl)-1,3,5-triazine, 2-(2-hydroxyphenyl)-4-(4-methoxyphenyl)-6-phenyl-1,3,5-triazine, 2-{2-hydroxy-4-[3-(2-ethylhexyl-1-oxy)-2-hydroxy-propyloxy]phenyl}-4,6-bis(2,4-dimethylphenyl)-1,3,5-triazine and 2-(2-hydroxy-4-(2-ethylhexyl)oxy)phenyl-4,6-di(4-phenyl)phenyl-1,3,5-triazine.

The effective absorption wavelength of benzophenone-based compounds is approximately 270 to 380 nm, and specific examples thereof include 2,4-dihydroxybenzophenone, 2-hydroxy-4-methoxybenzophenone, 2-hydroxy-4-octyloxybenzophenone, 2-hydroxy-4-decyloxybenzophenone, 2-hydroxy-4-dodecyloxybenzophenone, 2-hydroxy-4-benzyloxybenzophenone, 2-hydroxy-4-(2-hydroxy-3-methacryloxypropoxy)benzophenone, 2-hydroxy-4-methoxy-5-sulfobenzophenone, 2-hydroxy-4-methoxy-5-sulfobenzophenone trihydrate, 2-hydroxy-4-methoxy-2′-carboxybenzophenone, 2-hydroxy-4-octadecyloxybenzophenone, 2-hydroxy-4-diethylamino-2′-hexyloxycarbonylbenzophenone, 2,2′-dihydroxy-4-methoxybenzophenone, 2,2′,4,4′-tetrahydroxybenzophenone, 2,2′-dihydroxy-4,4′-dimethoxybenzophenone, and 1,4-bis(4-benzyloxy-3-hydroxyphenoxy)butane.

The salicylic acid-based compound above is preferably a compound having an effective absorption wavelength of approximately 290 to 330 nm, and typical examples thereof include phenyl salicylate, 4-t-butylphenyl salicylate, 4-octylphenyl salicylate, dibenzoylresorcinol, bis(4-t-butylbenzoyl)resorcinol, benzoylresorcinol, 2,4-di-t-butylphenyl 3,5-di-t-butyl-4-hydroxysalicylate, and hexadecyl 3,5-di-t-butyl-4-hydroxysalicylate.

The acrylate-based compound above is preferably a compound having an effective absorption wavelength of approximately 270 to 350 nm, and typical examples thereof include 2-ethylhexyl 2-cyano-3,3-diphenylacrylate, ethyl 2-cyano-3,3-diphenylacrylate, isooctyl 2-cyano-3,3-diphenylacrylate, hexadecyl 2-cyano-3-(4-methylphenyl)acrylate, methyl 2-cyano-3-methyl-3-(4-methoxyphenyl)cinnamate, butyl 2-cyano-3-methyl-3-(4-methoxyphenyl)cinnamate, methyl 2-carbomethoxy-3-(4-methoxyphenyl)cinnamate 2-cyano-3-(4-methylphenyl)acrylate salt, 1,3-bis(2′-cyano-3,3′-diphenylacryloyl)oxy)-2,2-bis(((2′-cyano-3,3′-diphenylacryloyl)oxy)methyl)propane, and N-(2-carbomethoxy-2-cyanovinyl)-2-methylindoline.

The oxalic diamide-based compound above is preferably a compound having an effective absorption wavelength of approximately 250 to 350 nm, and typical examples thereof include 4,4′-dioctyloxyoxanilide, 2,2′-dioctyloxy-5,5′-di-t-butyloxanilide, 2,2′-didodecyloxy-5,5′-di-t-butyloxanilide, 2-ethoxy-2′-ethyloxanilide, N,N′-bis(3-dimethylaminopropyl)oxamide, 2-ethoxy-5-t-butyl-2′-ethyloxanilide, and 2-ethoxy-2′-ethyl-5,4′-di-t-butyloxanilide.

The polymer material of the present invention may further contain a light stabilizer, or an antioxidant.

Preferable examples of the light stabilizer and the antioxidant include compounds described in JP-A-2004-117997. Specifically, compounds described on page 29, middle paragraph Nos. [0071] to [0111] of JP-A-2004-117997 are preferable. Especially, compounds represented by formula (TS-I), (TS-II), (TS-IV), or (TS-V) described on the paragraph No. [0072] are preferable.

The content of the ultraviolet absorbent comprising the compound represented by formula (1) or (2), in the polymer material according to the present invention, may vary according to the application and the usage of the polymer material and thus cannot be defined specifically, but can be determined easily by the person skilled in the art after some tests. It is preferably 0.001 to 10 mass %, more preferably 0.01 to 5 mass %, with respect to the total amount of the polymer material. The content of the ultraviolet absorbent other than the ultraviolet absorbent comprising the compound represented by formula (1) or (2) above can be determined properly according to the application of the present invention.

Although practically sufficient ultraviolet-shielding effect is obtained only with the ultraviolet absorbent in the present invention, a white pigment which has higher hiding power such as titanium oxide may be used for assurance. In addition, a trace (e.g. 0.05 mass % or less) amount of colorant may be used additionally, if the appearance or the color tone is of a problem or as needed. Alternatively, a fluorescent brightener may be used additionally for applications demanding transparency or whiteness. Examples of the fluorescent brighteners include commercialized products, the compounds described in JP-A-2002-53824, and the like.

Hereinafter, the polymer substance that can be used in the polymer material of the present invention will be described. An acrylic acid-based polymer, a polyester, a polycarbonate, or the blend thereof is preferably used as the polymer substance. Hereinafter, each of the polymers will be described in detail.

### (Acrylic Acid-Based Polymer)

The acrylic acid-based polymer, as used herein, is preferably a homopolymer or a copolymer obtained by polymerization of a compound represented by formula (A1) as the monomer component.

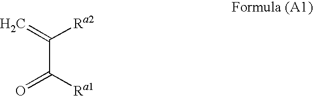

(In formula (A1), R^{a1} represents a hydroxyl group, a substituted or unsubstituted alkoxy group, a substituted or unsubstituted amino group, a substituted or unsubstituted alkyl group, a substituted or unsubstituted aryl group, or a substituted or unsubstituted heterocyclic group; R^{a2} represents a hydrogen atom, a methyl group, or an alkyl group having 2 or more carbon atoms.)

The formula (A1) will be described in detail.

In formula (A1), R^{a1} represents a hydroxyl group, a substituted or unsubstituted alkoxy group, a substituted or unsubstituted aryloxy group, a substituted or unsubstituted amino group, a substituted or unsubstituted alkyl group, a substituted or unsubstituted aryl group, or a substituted or unsubstituted heterocyclic group. Among these, R^{a1} is preferably a substituted or unsubstituted alkoxy group, or a substituted or unsubstituted aryloxy group; and particularly preferably a substituted or unsubstituted alkoxy group having 1 to 18 carbon atoms, or a substituted or unsubstituted aryloxy group having 6 to 24 carbon atoms.

R^{a2} represents a hydrogen atom, a methyl group, or an alkyl group having 2 or more carbon atoms. Among these, R^{a2} is preferably a hydrogen atom or a methyl group.

Thus, in preferable combination of the substituents of formula (A1), R^{a1} represents a substituted or unsubstituted alkoxy group having 1 to 18 carbon atoms or a substituted or unsubstituted aryloxy group having 6 to 24 carbon atoms, and R^{a2} represents a hydrogen atom or a methyl group.

Typical examples of the compound represented by formula (A1) include the followings:
acrylate derivatives such as methyl acrylate, ethyl acrylate, (n- or i-)propyl acrylate, (n-, i-, see- or t-)butyl acrylate, amyl acrylate, 2-ethylhexyl acrylate, dodecyl acrylate, chloroethyl acrylate, 2-hydroxyethyl acrylate, 2-hydroxypropyl acrylate, 2-hydroxypentyl acrylate, cyclohexyl acrylate, allyl acrylate, trimethylolpropane monoacrylate, pentaerythritol monoacrylate, benzyl acrylate, methoxybenzyl acrylate, chlorobenzyl acrylate, hydroxybenzyl acrylate, hydroxyphenethyl acrylate, dihydroxyphenethyl acrylate, furfuryl acrylate, tetrahydrofurfuryl acrylate, phenyl acrylate, hydroxyphenyl acrylate, chlorophenyl acrylate, sulfamoylphenyl acrylate, and 2-(hydroxyphenylcarbonyloxy)ethyl acrylate; methacrylate derivatives such as methyl methacrylate, ethyl methacrylate, (n- or i-)propyl methacrylate, (n-, i-, sec- or t-)butyl methacrylate, amyl methacrylate, 2-ethylhexyl methacrylate, dodecyl methacrylate, chloroethyl methacrylate, 2-hydroxyethyl methacrylate, 2-hydroxypropyl methacrylate, 2-hydroxypentyl methacrylate, cyclohexyl methacrylate, allyl methacrylate, trimethylolpropane monomethacrylate, pentaeiythritol monomethacrylate, benzyl methacrylate, methoxybenzyl methacrylate, chlorobenzyl methacrylate, hydroxybenzyl methacrylate, hydroxyphenethyl methacrylate, dihydroxyphenethyl methacrylate, furfuryl methacrylate, tetrahydrofurfuryl methacrylate, phenyl methacrylate, hydroxyphenyl methacrylate, chlorophenyl methacrylate, sulfamoylphenyl methacrylate, and 2-(hydroxyphenylcarbonyloxy)ethyl methacrylate; acrylamide derivatives such as acrylamide, N-methylacrylamide, N-ethylacrylamide, N-propylacrylamide, N-butylacrylamide, N-benzylacrylamide, N-hydroxyethylacrylamide, N-phenylacrylamide, N-tolylacrylamide, N-(hydroxyphenyl)acrylamide, N-(sulfamoylphenyl)acrylamide, N-(phenylsulfonyl)acrylamide, N-(tolylsulfonyl)acrylamide, N,N-dimethylacrylamide, N-methyl-N-phenylacrylamide, and N-hydroxyethyl-N-methylacrylamide; and methacrylamide derivatives such as methacrylamide, N-methylmethacrylamide, N-ethylmethacrylamide, N-propylmethacrylamide, N-butylmethacrylamide, N-benzylmethacrylamide, N-hydroxyethylmethacrylamide, N-phenylmethacrylamide, N-tolylmethacrylamide, N-(hydroxyphenyl)methacrylamide, N-(sulfamoylphenyl)methacrylamide, N-(phenylsulfonyl)methacrylamide, N-(tolylsulfonyl)methacrylamide, N,N-dimethylmethacrylamide, N-methyl-N-phenylmethacrylamide, and N-hydroxyethyl-N-methylmethacrylamide,

The acrylic acid-based polymer is preferably a single-component homopolymer obtained by polymerization of the monomer represented by formula (A1) above or a two-, three- or four-component, more preferably two- or three-component, copolymer prepared by polymerization using the monomer represented by formula (A1) above at a molar ratio of 10% to 90%, preferably 20% to 80% and also other monomer components or the other monomer components represented by formula (A1) above. Examples of the other monomer components include a substituted or unsubstituted styrene derivative, and acrylonitrile.

The acrylic acid-based polymer is preferably a homopolymer containing an acrylate or a methacrylate having 4 to 24 carbon atoms as the repeating unit or a two- or three-component copolymer containing an acrylate or a methacrylate as the repeating unit at a molar ratio of 10% to 90%.

### (Polyester)

Hereinafter, the polyester will be described. The polyester that can be used in the present invention contains the following dicarboxylic acid, the acid halide thereof or the following polyvalent carboxylic acid; and a diol as repeating units.

Examples of the dicarboxylic acid or the acid halides thereof include aliphatic or, alicyclic dicarboxylic acids such as adipic acid, superic acid, azelaic acid, sebacic acid, dodecanedioic acid, oxalic acid, malonic acid, succinic acid, glutaric acid, ethylsuccinic acid, pimelic acid, maleic acid, fumaric acid, itaconic acid, citraconic acid, mesaconic acid, 2-methylsuccinic acid, 2-methyladipic acid, 3-methyladipic acid, 3-methylpentanedioic acid, 2-methyloctanedioic acid, 3,8-dimethyldecanedioic acid, 3,7-dimethyldecanedioic acid, dimer acid, hydrogenated dimer acids, 1,2- or 1,3-cyclopentanedicarboxylic acids, and 1,2-, 1,3- or 1,4-cyclohexanedicarboxylic acids; and aromatic dicarboxylic acids such as phthalic acid, isophthalic acid, terephthalic acid, 1,4-naphthalenedicarboxylic acid, 2,5-naphthalenedicarboxylic acid, 2,6-naphthalenedicarboxylic acid, naphthalic acid, biphenyldicarboxylic acid, 2-methylisophthalic acid, 3-methylphthalic acid, 2-methylterephthalic acid, 2,4,5,6-tetramethylisophthalic acid, 3,4,5,6-tetramethylphthalic acid, 2-chloroterephthalic acid, 2-methylterephthalic acid, 5-methylisophthalic acid, 5-sodium sulfoisophthalic acid, 2,6-naphthalenedicarboxylic acid, hexahydroterephthalic acid, hexahydroisophthalic acid, 3-chloroisophthalic acid, 3-methoxyisophthalic acid, 2-fluoroisophthalic acid, 3-fluorophthalic acid, 2-fluoroterephthalic acid, 2,4,5,6-tetrafluoroisophthalic acid, 3,4,5,6-tetrafluorophthalic acid, 4,4′-oxybisbenzoic acid, 3,3′-oxybisbenzoic acid, 3,4′-oxybisbenzoic acid, 2,4′-oxybisbenzoic acid, 3,4′-oxybisbenzoic acid, 2,3′-oxybisbenzoic acid, 4,4′-oxybisoctafluorobenzoic acid, 3,3′-oxybisoctafluorobenzoic acid, 1,4-naphthalenedicarboxylic acid, 2,6-naphthalenedicarboxylic acid, 4,4′-biphenyldicarboxylic acid, 4,440 -diphenylethercarboxylic acid; and the like.

Examples of the polyvalent carboxylic acids other than the dicarboxylic acids include ethanetricarboxylic acid, propanetricarboxylic acid, butanetetracarboxylic acid, pyromellitic acid, trimellitic acid, trimesic acid, and 3,4,3′,4′-biphenyltetracarboxylic acid.

With respect to the polyester that can be used in the present invention, among these dicarboxylic acids and polyvalent carboxylic acid components, use of adipic acid, malonic acid, succinic acid, terephthalic acid, isophthalic acid, phthalic acid, 1,4-naphthalenedicarboxylic acid, 2,5-naphthalenedicarboxylic acid, 2,6-naphthalenedicarboxylic acid or trimellitic acid is preferable; and use of terephthalic acid, isophthalic acid, 1,4-naphthalenedicarboxylic acid, 2,5-naphthalenedicarboxylic acid or 2,6-naphthalenedicarboxylic acid is particularly preferable.

Examples of the diols include aliphatic glycols such as ethylene glycol, 1,2-propylene glycol, 1,3-propylene glycol, diethylene glycol, triethylene glycol, 1,2-butylene glycol, 1,3-butylene glycol, 2,3-butylene glycol, 1,4-butylene glycol, 1,5-pentanediol, neopentylglycol, 1,6-hexanediol, 1,2-cyclohexanediol, 1,3-cyclohexanediol, 1,4-cyclohexanediol, 1,2-cyclohexanedimethanol, 1,3-cyclohexanedimethanol, 1,4-cyclohexanedimethanol, 1,4-cyclohexanediethanol, 1,10-decamethylene glycol, 1,12-dodecanediol, polyethylene glycol, polytrimethylene glycol, and polytetramethylene glycol; aromatic glycols such as hydroquinone, 4,4′-dihydroxybisphenol, 1,4-bis(β-hydroxyethoxy)benzene, 1,4-bis(β-hydroxyethoxyphenyl)sulfone, bis(p-hydroxyphenyl)ether, bis(p-hydroxyphenyl)sulfone, bis(p-hydroxyphenyl)methane, 1,2-bis(p-hydroxyphenyl)ethane, bisphenol A, bisphenol C, 2,5-naphthalenediol, and ethyleneoxide adducts of these glycols; and the like.

With respect to the polyester that can be used in the present invention, among these diol components, use of ethylene glycol, 1,3-propylene glycol, diethylene glycol, neopentylglycol, hydroquinone, 4,4′-dihydroxybisphenol or bisphenol A is preferable; and use of ethylene glycol or 4,4′-dihydroxybisphenol is particularly preferable.

Specifically, preferable combinations of monomers and preferable polymers in the polyester that can be used in the present invention include polyethylene terephthalate prepared by using terephthalic acid as the dicarboxylic acid component and ethylene glycol as the diol component, polybutylene terephthalate prepared by using terephthalic acid as the dicarboxylic acid component and 1,4-butylene glycol as the diol component, and polyethylene naphthalate prepared by using 2,6-naphthalenedicarboxylic acid as the dicarboxylic acid component and ethylene glycol as the diol component.

### (Polycarbonate)

The polycarbonate that can be used in the present invention is prepared from the following polyvalent phenols and the following carbonates such as bisalkyl carbonate, bisaryl carbonate or phosgene.

Examples of the polyvalent phenols include hydroquinone, resorcin, 4,4′-dihydroxydiphenyl, bis(4-hydroxyphenyl)methane, 1,1-bis(4-hydroxyphenyl)ethane, 1,1-bis(4-hydroxyphenyl)-1-phenylethane, bisphenol A, bisphenol C, bisphenol E, bisphenol F, bisphenol M, bisphenol P, bisphenol S, bisphenol Z, 2,2-bis(3-methyl-4-hydroxyphenyl)propane, 1,1-bis(4-hydroxyphenyl)cyclohexane, 2,2-bis(3-phenyl-4-hydroxyphenyl)propane, 2,2-bis(3-isopropyl-4-hydroxyphenyl)propane, 2,2-bis(4-hydroxyphenyl)butane, 2,2-bis(3,5-dimethyl-4-hydroxyphenyl)propane, 2,2-bis(3,5-dibromo-4-hydroxyphenyl)propane, 4,4′-dihydroxydiphenylsulfone, 4,4′-dihydroxydiphenylsulfoxide, 4,4′-dihydroxydiphenylsulfide, 3,3′-dimethyl-4,4′-dihydroxydiphenylsulfide, and 4,4′-dihydroxydiphenyloxide.

With respect to the polycarbonate that can be used in the present invention, among these polyvalent phenol components, use of hydroquinone, resorcin, 4,4′-dihydroxydiphenyl or bisphenol A is preferable.

Examples of the carbonates include phosgene, diphenyl carbonate, bis(chlorophenyl)carbonate, dinaphthyl carbonate, bis(diphenyl)carbonate, dimethyl carbonate, diethyl carbonate, and dibutyl carbonate.

With respect to the polycarbonate that can be used in the present invention, among these carbonate components, use of phosgene, bis(diphenyl)carbonate, dimethyl carbonate, or diethyl carbonate is preferable.

Specifically, a preferable combination of monomers, i.e., a preferable polymer in the polycarbonate that can be used in the present invention is bisphenol A carbonate, which is prepared by using bisphenol A as the polyvalent phenol component and phosgene as the carbonate component.

Among the polymers above, polymethyl acrylate, polymethyl methacrylate, polyethylene terephthalate, polyethylene naphthalate, polybutylene terephthalate and polycarbonate are particularly preferable. Unexpectedly to the person skilled in the art, use of one of the preferable polymer substances resulted in drastic improvement in light fastness of the ultraviolet absorbent, compared to the ultraviolet absorbent prepared with a polymer substance other than those above.

The polymer substance for use in the present invention is preferably a thermoplastic resin.

The polymer substance for use in the present invention preferably has a transmittance of 80% or more. The transmittance in the present invention is the total light transmittance as determined according to the method described in the Chemical Society of Japan Ed., “Experimental Chemistry Lecture 29—Polymer materials,” 4th Ed., (Maruzen, 1992) p. 225 to 232.

The glass transition point (Tg) of the polymer substance for use in the present invention is preferably −80° C. or higher and 200° C. or lower, still more preferably −30° C. or higher and 180° C. or lower. In particular, a polyacrylate, a polycarbonate and a polyethylene terephthalate are preferable.

The polymer material prepared by using a polymer substance having a Tg in the range above gives a polymer material favorably in flexibility and hardness. When a polyacrylate, polycarbonate or polyethylene terephthalate is used, it leads to improvement in operational efficiency; and when the ultraviolet absorbent comprising the compound represented by formula (1) or (2) is used, it leads to improvement in the light fastness of the ultraviolet absorbent itself.

The polymer material according to the present invention may contain any additives such as antioxidant, photostabilizer, processing stabilizer, antidegradant, and compatibilizer, as needed in addition to the polymer substance above and the ultraviolet light inhibitor (absorbent).

The polymer material according to the present invention contains the polymer substance above. The polymer material according to the present invention may be made only of the above-described polymer substance, or may be formed by using the polymer substance dissolved in a solvent.

When the polyethylene terephthalate is used as the polymer substance, the polymer material according to the present invention is preferably produced by melt-kneading of the polyethylene terephthalate and the ultraviolet absorbent at a temperature of 200° C. or higher. Polymer materials prepared by the melt-kneading polyethylene terephthalate at the temperature or less possibly may give polymer materials containing the ultraviolet absorbent unevenly dispersed in the spot-like pattern.

The content of the ultraviolet absorbent in the polymer material according to the present invention is preferably 0.1 mass % to 50 mass %, more preferably 0.1 mass % to 25 mass %, and particularly preferably 0.4 mass % to 10 mass %, with respect to 100 mass % of the polyethylene terephthalate. A too low content of the ultraviolet absorbent may result in production of a polymer material that does not absorb the light in the ultraviolet region completely, because of insufficiency of the ultraviolet absorbent added.

The compound represented by formula (1) or (2) for use in the present invention, which is superior in solubility, gives a polymer material easily, as it is dissolved in a various solvent with a polymer and the solution coated. In preparation of the polymer material, a plasticizer may not be added. In addition, a polymer material prepared by solvent coating or polymer kneading has an advantage that it is superior in light fastness, compared to the polymer material prepared by using a plasticizer.

The compound represented by formula (1) or (2) mostly have a molecular weight of 1000 or less, and thus, the idea of using such a compound as it is melted under an environment at high temperature for prolonged period, for example during PET kneading, which may lead to volatilization and decomposition, was not easily conceived by the person skilled in the art.

When the acrylate or the polycarbonate is used as the polymer substance, the polymer material according to the present invention is preferably prepared by dissolving the polyacrylate or polycarbonate and the ultraviolet absorbent in a solvent having a boiling point of 200° C. or lower and coating the resulting solution on a base plate. If a solvent having a boiling point of 200° C. or higher is used, it is needed to volatilize the solvent at high temperature, which may make the processing step more complicated.

The content of the ultraviolet absorbent in the polymer material according to the present invention, is preferably 0.1 mass % to 50 mass %, more preferably 0.1 mass % to 25 mass %, and particularly more preferably 0.4 mass % to 10 mass %, with respect to 100 mass % of the polyacrylate or the polycarbonate. When the added amount is too low, polymer materials absorbing the light in the entire ultraviolet region may not be produced, because of insufficiency of the ultraviolet absorbent added.

The solvent having a boiling point of 200° C. or less that can be used in the present invention is not particularly limited, as long as the solvent is able to dissolve or disperse the ultraviolet absorbent of the present invention. The boiling point of the solvent is preferably in the range of 0° C. to 200° C., more preferably from 20° C. to 150° C., and furthermore preferably from 30° C. to 120° C., from viewpoints of the coated surface state and drying of the solvent after coating. Examples of the solvent include alcoholic solvents (e.g., methanol, ethanol, isopropanol, and tetrafluoropropanol), halogen-series solvents (e.g., methylene chloride, chloroform, chlorobenzene, and dichlorobenzene), ketone-series solvents (e.g., acetone, ethylmethylketone, and cyclohexanone), hydrocarbon-series solvents (e.g., benzene, toluene, and cyclohexane), ester-series solvents (e.g., ethyl acetate, and butyl acetate), and ether-series solvents (e.g., dioxane, and tetrahydrofuran). If necessary, these solvents may be used in combination of two or more kinds.

Examples of the substrate that can be used in the present invention include inorganic substrates such as a glass substrate, an iron substrate, an aluminum substrate, a silicon substrate, and a ceramic substrate; and polymer material substrates such as a polyethylene terephthalate (PET) film substrate, a triacetyl cellulose (TAC) film substrate, or a polycarbonate film substrate. The form of these substrates may be various forms such as a plate-like, sheet-like, or disc-like shape. That is, any shape of substrate may be used, as long as the shape does not prevent a polymer material from being coated.

The polymer material according to the present invention is applicable to any application where synthetic resin is used, and particularly favorably to applications where there is possibility of exposure to light such as sunlight or ultraviolet light. Specific examples thereof include glass alternatives and their surface-coating material; coating agents for the window glass, lighting glass and light source-protecting glass such as of house, facility, and vehicle; interior and exterior materials such as of house, facility and vehicle, paints for the interior and exterior materials; materials for ultraviolet-emission sources such as fluorescent lamp and mercury lamp; materials for precision machines and electric and electronic devices; materials for shielding electromagnetic and other waves emitted from various displays; containers and packaging materials such as of food, chemicals, and medicine; discoloration inhibitors for agricultural and industrial sheet or film, print, colored products, dyes and pigments; cosmetics such as anti-sunburn cream, shampoo, rinse, and hair dressing; apparel fiber products such as sport wear, stockings and cap and the fibers; home interior products such as curtain, carpet and wall paper; medical devices such as plastic lens, contact lens and artificial eye; optical materials such as optical filter, prism, mirror, and photographic material; stationery products such as tape and ink; display plates and devices and the surface-coating materials thereof, and the like. Alternatively, the polymer material according to the present invention may be used in cosmetic applications.

The shape (form) of the polymer material according to the present invention may be flat film, powder, spherical particle, crushed particle, bulky continuous particle, fiber, solenoid, hollow fiber, granule, plate, porous particle, or the other.

The polymer material according to the present invention, which contains the ultraviolet absorbent comprising the compound represented by formula (1) or (2), is superior in light resistance (ultraviolet fastness), causing no precipitation or bleed-out of the ultraviolet absorbent during long-term use. In addition, the polymer material according to the present invention, which has superior long-wavelength ultraviolet absorption capacity, can be used as an ultraviolet-absorbing filter or container, for protection, for example, of an ultraviolet-sensitive compound therein. It is possible to obtain a molded article (such as container) of the polymer material according to the present invention, for example, by molding the polymer substance by any molding method such as extrusion molding or injection molding. It is also possible to prepare a molded article coated with an ultraviolet-absorbing film made of the polymer material according to the present invention, by coating and drying a solution of the polymer substance on a separately prepared molded article.

When the polymer material according to the present invention is used as an ultraviolet-absorbing filter or film, the polymer substance is preferably transparent. Examples of the transparent polymer materials include polycarbonate, polyesters (e.g., polyethylene terephthalate, polyethylene naphthalate, polybutylene terephthalate, poly-1,4-cyclohexane dimethylene terephthalate, polyethylene 1,2-diphenoxyethane-4,4′-dicarboxylate, polybutylene terephthalate), and polymethyl methacrylate. Preferable are polycarbonate, polyethylene terephthalate, and acrylic resins. The polymer material according to the present invention may be used as a transparent support, and the transmittance of the transparent support in such a case is preferably 80% or more, more preferably 86% or more.

Hereinafter, the packaging material containing the polymer material according to the present invention will be described. The packaging material containing the polymer material according to the present invention may be a packaging material of any kind of polymer, as long as it contains the ultraviolet absorbent comprising the compound represented by formula (1) or (2). Examples thereof include the thermoplastic resins described in JP-A-8-208765; the polyesters described in JP-A-10-168292 and JP-A-2004-285189; and the heat-shrinkable polyesters described in JP-A-2001-323082. It may be, for example, the paper coated with a resin containing an ultraviolet absorbent described in JP-A-2006-240734.

The packaging material containing the polymer material according to the present invention may be that for packaging anything such as food, beverage, medicine, cosmetics, or individual health care product. Examples thereof include the food packaging materials described in JP-A-11-34261 and JP-A-2003-237825; the colored liquid packaging materials described in JP-A-8-80928; the liquid preparation-packaging materials described in JP-A-2004-51174; the medicine container packaging materials described in JP-A-8-301363 and JP-A-1 1-276550; the medical sterilization packaging materials described in JP-A-2006-271781; the photographic photosensitive material packaging materials described in JP-A-7-287353; the photograph film packaging materials described in JP-A-2000-56433; the UV-hardening ink packaging materials described in JP-A-2005-178832; the shrink labels described in JP-A-2003-200966 and JP-A-2006-323339; and the like.

The packaging material containing the polymer material according to the present invention may be the transparent packaging material described, for example, in JP-A-2004-51174 or the light-shielding packaging material described, for example, in JP-A-2006-224317.

The packaging material containing the polymer material according to the present invention may have ultraviolet light-shielding property as well as other properties, as described, for example, in JP-A-2001-26081 and JP-A-2005-305745. Examples thereof include the packaging materials having gas-barrier property described, for example, in JP-A-2002-160321; those containing an oxygen indicator as described, for example, in JP-A-2005-156220; those containing both an ultraviolet absorbent and a fluorescent brightener described, for example, in JP-A-2005-146278; and the like.

The packaging material containing the polymer material according to the present invention may be prepared by any method. Examples of the method include the method of forming an ink layer described, for example, in JP-A-2006-130807; the method of melt-extruding and laminating a resin containing an ultraviolet absorbent described, for example, in JP-A-2001-323082 and JP-A-2005-305745; the method of coating on a base film described, for example, in JP-A-9-142539; the method of dispersing an ultraviolet absorbent in an adhesive described, for example, in JP-A-9-157626; and the like.

Hereinafter, the container containing the polymer material according to the present invention will be described. The container containing the polymer material according to the present invention may be a container of any kind of polymer, as long as it contains the ultraviolet absorbent comprising the compound represented by formula (1) or (2). Examples thereof include the thermoplastic resin containers described in JP-A-8-324572; the polyester containers described in JP-A-2001-48153, JP-A-2005-105004, and JP-A-2006-1568; the polyethylene naphthalate containers described in JP-A-2000-238857; and the like.

The container containing the polymer material according to the present invention is used as containers in various applications including food, beverage, medicine, cosmetics, individual health care product, shampoo and the like. Examples thereof include the liquid fuel-storing containers described in JP-A-5-139434; the golf ball containers described in JP-A-7-289665; the food containers described in JP-A-9-295664 and JP-A-2003-237825; the liquor containers described in JP-A-9-58687; the medicine-filling containers described in JP-A-8-155007; the beverage containers described in JP-A-8-324572 and JP-A-2006-298456; the oily food containers described in JP-A-9-86570; the analytical reagent solution containers described in JP-A-9-113494; the instant noodle containers described in JP-A-9-239910; the light-resistant cosmetic preparation containers described in JP-A-11-180474, JP-A-2002-68322, and JP-A-2005-278678; the medicine containers described in JP-A-11-276550; the high-purity chemical solution containers described in JP-A-11-290420; the liquid agent containers described in JP-A-2001-106218; the UV-hardening ink containers described in JP-A-2005-178832; the plastic ampoules described in WO 04/93775 pamphlet; and the like.

The container containing the polymer material according to the present invention may have ultraviolet-shielding property as well as other properties, as described, for example, in JP-A-5-305975 and JP-A-7-40954. Examples of such containers include the antimicrobial containers described in JP-A-10-237312; the flexuous containers described in JP-A-2000-152974; the dispenser containers described in JP-A-2002-264979; the biodegradable containers described in, for example, JP-A-2005-255736; and the like.

The container containing the polymer material according to the present invention may be prepared by any method. Examples of the method include the two-layer stretching blow-molding method described in JP-A-2002-370723; the multilayer coextrusion blow-molding method described in JP-A-2001-88815; the method of forming an ultraviolet-absorbing layer on the external surface of an container described in JP-A-9-241407; the methods of using a shrinkable film described in JP-A-8-91385, JP-A-9-48935, JP-T-11-514387, JP-A-2000-66603, JP-A-2001-323082, JP-A-2005-105032, and WO 99/29490 pamphlet; the method of using a supercritical fluid described in JP-A-11-255925; and the like.

Hereinafter, the paint and the coated film containing the polymer material according to the present invention will be described. The paint containing the polymer material according to the present invention may be a paint of any composition, as long as it contains the ultraviolet absorbent comprising the compound represented by formula (1) or (2). Examples thereof include those of acrylic resin-base, and polyester resin-base. To these resins, a base compound, curing agent, diluent, leveling agent, cissing inhibitor or the like may be added.

For example, when an acrylic urethane resin or a silicon acrylic resin is selected as the transparent resin component, the curing agent is preferably polyisocyanate; and the diluent is preferably a hydrocarbon-based solvent such as toluene or xylene, an ester-based solvent such as isobutyl acetate, butyl acetate and amyl acetate, or an alcohol-based solvent such as isopropyl alcohol or butyl alcohol. The acrylic urethane resin is an acrylic urethane resin obtained by reaction of a methacrylate (typically, methyl methacrylate), hydroxyethyl methacrylate copolymer and a polyisocyanate. In such a case, the polyisocyanate is, for example, tolylene diisocyanate, diphenylmethane diisocyanate, polymethylene polyphenylene polyisocyanate, tolidine diisocyanate, naphthalene diisocyanate, hexamethylene diisocyanate, isophorone diisocyanate, xylylene diisocyanate, dicyclohexylmethane diisocyanate, hexamethylene diisocyanate or the like. Examples of other transparent resin components include polymethyl methacrylate, polymethyl methacrylate/styrene copolymer, and the like. In addition to these components, a leveling agent such as an acrylic or silicone resin, a cissing inhibitor such as a silicone-based or acrylic inhibitor, and others may be added as needed.

The paint containing the polymer material according to the present invention may be used in any application. Examples thereof include the ultraviolet-shielding paints described in JP-A-7-26177, JP-A-9-169950, JP-A-9-221631, and JP-A-2002-80788; the ultraviolet-infrared-shielding paints described in JP-A-10-88039; the electromagnetic wave-shielding paints described in JP-A-2001-55541; the clear paints described in JP-A-8-81643; the metallic paint compositions described in JP-A-2000-186234; the cation electrodeposition paints described in JP-A-7-166112; the antimicrobial and lead-free cation electrodeposition paints described in JP-A-2002-294165; the powder paints described in JP-A-2000-273362, JP-A-2001-279189, and JP-A-2002-271227; the aqueous intermediate-layer paints, aqueous metallic paints, and aqueous clear paints described in JP-A-2001-9357; the topcoat paints for automobile, construction, and civil work described in JP-A-2001-316630; the hardening paints described in JP-A-2002-356655; the coat-film forming compositions for use on plastic materials such as automobile bumper described in JP-A-2004-937; the paints for a metal plate described in JP-A-2004-2700; the hardening gradient coat films described in JP-A-2004-169182; the coating materials for an electric wire described in JP-A-2004-107700; the paints for automobile repair described in JP-A-6-49368; the anionic electrodeposition paints described in JP-A-2002-38084 and JP-A-2005-307161; the paints for an automobile described in JP-A-5-78606, JP-A-5-185031, JP-A-10-140089, JP-T-2000-509082, JP-T-2004-520284, and WO 2006/097201 pamphlet; the paints for a coated steel plate described in JP-A-6-1945; the paints for a stainless steel described in JP-A-6-313148; the lamp moth-repellent paints described in JP-A-7-3189; the UV-hardening paints described in JP-A-7-82454; the antimicrobial paints described in JP-A-7-118576; the eyestrain protection paints described in JP-A-2004-217727; the anti-fog paints described in JP-A-2005-314495; the ultra-weather-resistance paints described in JP-A-10-298493; the gradient paints described in JP-A-9-241534; the photocatalyst paints described in JP-A-2002-235028; the strippable paints described in JP-A-2000-345109; the concrete separation paints described in JP-A-6-346022; the anti-corrosion paints described in JP-A-2002-167545; the protective paints described in JP-A-8-324576; the water-repellent protective paints described in JP-A-9-12924; the anti-plate glass scattering paints described in JP-A-9-157581; the alkali-soluble protective paints described in JP-A-9-59539; the aqueous temporary protective paint compositions described in JP-A-2001-181558; the flooring paints described in JP-A-10-183057; the emulsion paints described in JP-A-2001-115080; the two-liquid aqueous paints described in JP-A-2001-262056; the one-liquid paints described in JP-A-9-263729; the UV-hardening paints described in JP-A-2001-288410; the electron beam-hardening paint compositions described in JP-A-2002-69331; the thermosetting paint compositions described in JP-A-2002-80781; the aqueous paints for baking lacquer described in JP-T-2003-525325; the powder paints and the slurry paints described in JP-A-2004-162021; the repair paints described in JP-A-2006-233010; the powder-paint aqueous dispersions described in JP-T-11-514689; the paints for a plastic article described in JP-A-2001-59068 and JP-A-2006-160847; the electron beam-hardening paints described in JP-A-2002-693 31; and the like.

The paint containing the polymer material according to the present invention generally contains a paint (containing a transparent resin component as the principal component) and an ultraviolet absorbent comprising the compound represented by formula (1) or (2). The paint contains the ultraviolet absorbent preferably in an amount of 0 to 20 mass % with respect to the resin. The thickness of the film coated is preferably 2 to 1,000 μm, more preferably 5 to 200 μm. The method of coating the paint is arbitrary, and examples of the method include a spray method, a dipping method, a roller coating method, a flow coater method, a blow coating method, and the like. The dry after coating is preferably carried out at a temperature of approximately room temperature to 120° C. for 10 to 90 minutes, although the condition may vary according to the paint composition.

The coated film containing the polymer material according to the present invention is a coated film formed by using the paint containing the polymer material according to the present invention that contains the ultraviolet absorbent comprising the compound represented by formula (1) or (2).

Hereinafter, the ink containing the polymer material according to the present invention will be described. The ink containing the polymer material according to the present invention may be any ink in any form, as long as it contains the ultraviolet absorbent comprising the compound represented by formula (1) or (2). For example, it may be dye ink, pigment ink, water-based ink, oil-based ink, or the like. It may be used in any application. Examples of the applications include the screen printing ink described in JP-A-8-3502; the flexographic printing ink described in JP-T-2006-521941; the gravure printing ink described in JP-T-2005-533915; the lithographic offset printing ink described in JP-T-11-504954; the letterpress printing ink described in JP-T-2005-533915; the UV ink described in JP-A-5-254277; the EB ink described in JP-A-2006-30596; and the like. Other examples thereof include the inkjet inks described in JP-A-11-199808, WO 99/67337 pamphlet, JP-A-2005-325150, JP-A-2005-350559, JP-A-2006-8811, and JP-T-2006-514130; the photochromic ink described in JP-A-2006-257165; the thermal transfer ink described in JP-A-8-108650; the masking ink described in JP-A-2005-23111; the fluorescence ink described in JP-A-2004-75888; the security ink described in JP-A-7-164729; the DNA ink described in JP-A-2006-22300; and the like.

Any product obtained by using the ink containing the polymer material according to the present invention is also included in the present invention. Examples thereof include the print described in JP-A-2006-70190, and laminated films obtained by laminating the print, and the packaging materials and containers prepared by using the laminated film; the ink-receiving layer described in JP-A-2002-127596; and the like.

Hereinafter, the fiber containing the polymer material according to the present invention will be described. The fiber containing the polymer material according to the present invention may be a fiber of any kind of polymer, as long as it contains the ultraviolet absorbent comprising the compound represented by formula (1) or (2). Examples thereof include the polyester fibers described in JP-A-5-117508, JP-A-7-119036, JP-A-7-196631, JP-A-8-188921, JP-A-10-237760, JP-A-2000-54287, JP-A-2006-299428, and JP-A-2006-299438; and the like.

The fiber containing the polymer material according to the present invention may be prepared by any method. Examples of the method include the method, as described in JP-A-6-228818, of processing a polymer previously containing the ultraviolet absorbent comprising the compound represented by formula (1) or (2) into fiber, and the methods, as described, for example, in JP-A-5-9870, JP-A-8-188921, and JP-A-10-1587, of processing a material processed in a fiber form with a solution containing the ultraviolet absorbent comprising the compound represented by formula (1) or (2). As described in JP-A-2002-212884 and JP-A-2006-16710, the fiber may be prepared by using a supercritical fluid.

The fiber containing the polymer material according to the present invention can be used in various applications. Examples thereof include the clothing described in JP-A-5-148703; the lining described in JP-A-2004-285516; the underwear described in JP-A-2004-285517; the blanket described in JP-A-2003-339503; the hosiery described in JP-A-2004-11062; the synthetic leather described in JP-A-11-302982; the moth-repellent mesh sheet described in JP-A-7-289097; the mesh sheet for construction described in JP-A-10-1868; the carpet described in JP-A-5-256464; the moisture-permeable water-repellent sheet described in JP-A-5-193037; the nonwoven fabric described in JP-A-6-114991; the ultrafine fiber described in JP-A-11-247028; the fibrous sheet described in JP-A-2000-144583; the refreshing clothing described in JP-A-5-148703; the moisture-permeable water-repellent sheet described in JP-A-5-193037; the flame-resistant synthetic suede cloth structure described in JP-A-7-18584; the resin tarpaulin described in JP-A-8-41785; the filming agent, external wall material, and agricultural greenhouse described in JP-A-8-193136; the net and mesh for construction described in JP-A-8-269850; the filter substrate described in JP-A-8-284063; the stainproof filming agent described in JP-A-9-57889; the mesh fabric and land net described in JP-A-9-137335; the underwater net described in JP-A-10-165045; the ultrafine fibers described in JP-A-11-247027 and 11-247028; the textile fiber described in JP-A-7-310283 and JP-T-2003-528974; the air-bag base cloth described in JP-A-2001-30861; the ultraviolet-absorbing fiber products described in JP-A-7-324283, JP-A-8-20579, and JP-A-2003-147617; and the like.

Hereinafter, the construction material containing the polymer material according to the present invention will be described. The construction material containing the polymer material according to the present invention may be a construction material of any kind of polymer, as long as it contains the ultraviolet absorbent comprising the compound represented by formula (1) or (2). Examples thereof include the polyester-based material described in JP-A-2002-161158; the polycarbonate-based material described in JP-A-2003-160724; and the like.

The construction material containing the polymer material according to the present invention may be prepared by any method. Examples of the method include the method, as described in JP-A-8-269850, of forming a material containing the ultraviolet absorbent comprising the compound represented by formula (1) or (2) into a desired shape; the methods, as described, for example, in JP-A-10-205056, of forming a laminate of a material containing the ultraviolet absorbent comprising the compound represented by formula (1) or (2); the methods, as described, for example, in JP-A-8-151457, of forming a coated layer containing the ultraviolet absorbent comprising the compound represented by formula (1) or (2); and the methods, as described, for example, in JP-A-2001-172531, of forming it by coating a paint containing the ultraviolet absorbent comprising the compound represented by formula (1) or (2).

The construction material containing the polymer material according to the present invention can be used in various applications. Examples thereof include the external construction materials described in JP-A-7-3955, JP-A-8-151457, and JP-A-2006-266042; the wood structure for construction described in JP-A-8-197511; the roofing material for construction described in JP-A-9-183159; the antimicrobial construction material described in JP-A-11-236734; the base construction material described in JP-A-10-205056; the antifouling construction material described in JP-A-11-300880; the flame-resistant material described in JP-A-2001-9811; the ceramic construction material described in JP-A-2001-172531; the decorative construction material described in JP-A-2003-328523; the painted products for construction described in JP-A-2002-226764; the facing materials described in JP-A-10-6451, JP-A-10-16152, and JP-A-2006-306020; the construction net described in JP-A-8-269850; the moisture-permeable water-repellent sheet for construction described in JP-A-9-277414; the mesh sheet for construction described in JP-A-10-1868; the construction film described in JP-A-7-269016; the decorative film described in JP-A-2003-211538; the coating materials for construction described in JP-A-9-239921, JP-A-9-254345, and JP-A-10-44352; the adhesive composition for construction described in JP-A-8-73825; the civil work construction structure described in JP-A-8-207218; the pathway coating material described in JP-A-2003-82608; the sheet-shaped photocuring resin described in JP-A-2001-139700; the wood-protecting paint described in JP-A-5-253559; the push-switch cover described in JP-A-2005-2941780; the bond-sheeting agent described in JP-A-9-183159; the base construction material described in JP-A-10-44352; the wall paper described in JP-A-2000-226778; the decorative polyester film described in JP-A-2003-211538; the decorative polyester film for molding described in JP-A-2003-211606; the flooring material described in JP-A-2004-3191; and the like.

Hereinafter, the recording medium containing the polymer material according to the present invention will be described. The recording medium containing the polymer material according to the present invention may be any medium, as long as it contains the ultraviolet absorbent comprising the compound represented by formula (1) or (2). Examples thereof include the inkjet recording media described in JP-A-9-309260, JP-A-2002-178625, JP-A-2002-212237, JP-A-2003-266926, JP-A-2003-266927, and JP-A-2004-181813; the image-receiving medium for thermal transfer ink described in JP-A-8-108650; the image-receiving sheet for sublimation transfer described in JP-A-10-203033; the image-recording medium described in JP-A-2001-249430; the heat-sensitive recording medium described in JP-A-8-258415; the reversible heat-sensitive recording media described in JP-A-9-95055, JP-A-2003-145949, and JP-A-2006-167996; the information-photorecording medium described in JP-A-2002-367227; and the like.

Hereinafter, the image display device containing the polymer material according to the present invention will be described. The image display device containing the polymer material according to the present invention may be any device, as long as it contains the ultraviolet absorbent comprising the compound represented by formula (1) or (2). Examples thereof include the image display device employing an electrochromic element described in JP-A-2006-301268; the image display device of so-called electronic paper described in JP-A-2006-293155; the plasma display described in JP-A-9-306344; the image display device employing an organic EL element described in JP-A-2000-223271; and the like. The ultraviolet absorbent comprising the compound represented by formula (1) or (2) according to the present invention may be contained, for example, in the ultraviolet-absorbing layer formed in the laminated structure described in JP-A-2000-223271 or in a suitable part such as the circularly polarizing plate described, for example, in JP-A-2005-189645.

Hereinafter, the solar cell cover containing the polymer material according to the present invention will be described. The solar cell may be any kind of solar cell such as crystalline silicon solar cell, amorphous silicon solar cell, or dye-sensitized solar cell. As described in JP-A-2000-174296, a cover material has been used as a part for providing a crystalline silicon solar cell or an amorphous silicon solar cell with antifouling property, impact resistance, and durability. As described in JP-A-2006-282970, dye-sensitized solar batteries, which employ a metal oxide-based semiconductor that is activated by excitation of light (in particular, ultraviolet light) as its electrode material, have a problem of the photosensitizer colorant adsorbed being decomposed and thus the photovoltaic efficiency gradually declining, and for that reason, installation of an additional ultraviolet-absorbing layer was proposed.

The solar cell cover containing the polymer material according to the present invention may be a cover of any kind of polymer. Examples of the polymer include the polyester described in JP-A-2006-3 10461; the acrylic resin described in JP-A-2004-227843; and the like.

The solar cell cover containing the polymer material according to the present invention may be prepared by any method. For example, the ultraviolet-absorbing layer described in JP-A-11-40833 may be formed; the layers respectively containing the ultraviolet absorbent may be laminated, as described in JP-A-2005-129926; it may be contained in the filler layer resin, as described in JP-A-2000-91611; or a film may be formed, together with the ultraviolet absorbent-containing polymer described in JP-A-2005-346999.

The solar cell cover containing the polymer material according to the present invention may be in any form. Examples thereof include the film and sheet described in JP-A-2000-91610 and JP-A-11-261085; the laminate film described, for example, in JP-A-11-40833; the cover glass structure described in JP-A-11-214736; and the like. The ultraviolet absorbent may be contained in the sealer described in JP-A-2001-261904.

A glass-coating film and glass using the same containing the ultraviolet absorbent comprising the compound represented by formula (1) or (2) will be described. The glass and the glass-coating film may be any one in any form, so long as they contain the ultraviolet absorbent comprising the compound represented by formula (1) or (2). Further, they may be used for any purposes. Examples thereof include a heat ray-blocking (barrier) glass described in JP-A-5-58670 and JP-A-9-52738; a window glass described in JP-A-7-48145; a colored glass described in JP-A-8-157232, JP-A-10-45425 and JP-A-11-217234; an ultraviolet sharp-cut glass for high intensity light sources such as mercury lamp and metal halide lamp described in JP-A-8-59289; a frit glass described in JP-A-5-43266; an ultraviolet-blocking (barrier) glass for vehicles described in JP-A-5-163174; a colored heat ray-absorbing glass described in JP-A-5-270855; a fluorescent brightening agent-containing ultraviolet-absorbing insulation glass described in JP-A-6-316443; an ultraviolet and heat ray-blocking (barrier) glass for automobiles described in JP-A-7-237936; a cladding stained glass described in JP-A-7-267682; a water repellent ultraviolet and infrared ray-blocking (barrier) glass described in JP-A-7-291667; a glass for head up display of vehicles described in JP-A-7-257227; a dimming heat barrier multilayer window described in JP-A-7-232938; an ultraviolet and infrared rays cut glass described in JP-A-5-78147, JP-A-5-61835 and JP-A-8-217486; an ultraviolet ray cut glass described in JP-A-6-127974 and JP-A-7-53241; an ultraviolet and infrared rays-absorbing window glass described in JP-A-8-165146; an ultraviolet cut-off antifouling window film described in JP-A-10-17336; a light transmission panel for plantation house described in JP-A-9-67148; an ultraviolet and infrared rays-absorbing and low transmission glass described in JP-A-10-114540; a low reflectance and low permeability glass described in JP-A-11-302037; an edge-light apparatus described in JP-A-2000-16171; a rough surface-formed plate glass described in JP-A-2000-44286; a laminated display glass described in JP-A-2000-103655; a conductive coating glass described in JP-A-2000-133987; an anti-glare glass described in JP-A-2000-191346; an ultraviolet and infrared rays-absorbing and middle transmission glass described in JP-A-2000-7371; a privacy-protected window glass for vehicles described in JP-A-2000-143288; an anti-fogged glass for vehicles described in JP-A-2000-239045; a glass for paving materials described in JP-A-2001-287977; a drain anti-adhesion and heat ray-blocking glass plate described in JP-A-2002-127310; an ultraviolet and infrared rays-absorbing bronze glass described in JP-A-2003-342040; a glass described in WO 01/019748; a glass with ID identification function described in JP-A-2004-43212; a PDP optical filter described in JP-A-2005-70724; and a garret window described in JP-A-2005-105751. The glass-coating film containing the ultraviolet absorbent comprising the compound represented by formula (1) or (2) and the glass using the film may be produced according to any method.

Other examples of applications include the illumination light source covers described in JP-A-8-102296, 2000-67629, and JP-A-2005-353554; the synthetic leathers described in JP-A-5-272076 and JP-A-2003-239181; the sport goggle described in JP-A-2006-63162; the deflection lens described in JP-A-2007-93649; the hard-coat film for various plastic products described in JP-A-2001-214121, JP-A-2001-214122, JP-A-2001-315263, JP-A-2003-206422, JP-A-2003-25478, JP-A-2004-137457, and JP-A-2005-132999; the hard-coat film for bonding on external window described in JP-A-2002-36441; the window film described in JP-A-10-250004; the high-definition antiglare hard-coat film described in JP-A-2002-36452; the antistatic hard-coat film described in JP-A-2003-39607; the permeable hard-coat film described in JP-A-2004-114355; the antiforgery recoding media described in JP-A-2002-113937; the turf purpura-preventing agent described in JP-A-2002-293706; the resin film/sheet-bonding sealant described in JP-A-2006-274179; the optical parts described in JP-A-2005-326761; the rubber-coating agent described in JP-A-2006-335855; the agricultural covering materials described in JP-A-10-34841 and JP-A-2002-114879; the color candles described in JP-T-2004-532306 and JP-T-2004-530024; the cloth-rinsing agent composition described in JP-T-2004-525273; the prism sheet described in JP-A-10-287804; the protective layer transfer sheet described in JP-A-2000-71626; the photocuring resin product described in JP-A-2001-139700; the flooring sheet described in JP-A-2001-159228; the light-blocking printing label described in JP-A-2002-189415; the fuel cup described in JP-A-2002-130591; the articles with hard-coat film described in JP-A-2002-307619; the intermediate transfer recording medium described in JP-A-2002-307845; the synthetic hair described in JP-A-2006-316395; the low-temperature heat-shrinkable films for label described in WO 99/29490 pamphlet and JP-A-2004-352847; the fishing goods described in JP-A-2000-224942; the micro beads described in JP-A-8-208976; the precoated metal plate described in JP-A-8-318592; the thin film described in JP-A-2005-504735; the heat-shrinkable film described in JP-A-2005-105032; the in-mold molding label described in JP-A-2005-37642; the projection screen described in JP-A-2005-55615; the decorative sheets described in JP-A-9-300537, JP-A-2000-25180, JP-A-2003-19776, and JP-A-2005-74735; the hot-melt adhesive described in JP-A-2001-207144; the adhesives described in JP-T-2002-543265, JP-T-2002-543266 and U.S. Pat. No. 6,225,384; the electrodeposition coat and the basecoat described in JP-A-2004-352783; the wood surface-protecting agent described in JP-A-7-268253; the light-controlling materials, light-controlling films, and light-controlling glasses described in JP-A-2003-253265, JP-A-2005-105131, JP-A-2005-300962, and Japanese Patent No. 3915339; the moth-repellent lamp described in JP-A-2005-304340; the touch panel described in JP-A-2005-44154; the sealant for bonding resin film sheet described in JP-A-2006-274197; the polycarbonate film coating material described in JP-A-2006-89697; the optical fiber tape described in JP-A-2000-231044; the solid wax described in JP-T-2002-527559; and the like.

Hereinafter, the method of evaluating the light stability of the polymer material will be described. Preferable methods of evaluating the light stability of the polymer material are described, for example, in “Methods for Improving the Photostability of Polymers” (CMC Publishing, 2000) p. 85 to 107; “Basis and Physical Properties of High Functional Coatings” (CMC Publishing, 2003), p. 314 to 359; “Durability of Polymer Materials and Composite Material Products” (CMC Publishing, 2005); “Elongation of Lifetime of Polymer Materials and Environmental Measures” (CMC Publishing, 2000); H. Zweifel Ed., “Plastics Additives Handbook, 5th Edition” (Hanser Publishers), p. 238 to 244; and Tadahiko Kutsura, “Basic Seminar 2. Science of Plastic Packaging Container” (Society of packaging Science & Technology, Japan, 2003), Chapter 8.

In addition, the light stability in each application can be evaluated by the following known evaluation methods.

The photodegradation of polymer materials can be determined by the method described in JIS-K7105:1981, JIS-K7101:1981, JIS-K7102:1981, JIS-K7219:1998, JIS-K7350-1:1995, JIS-K7350-2:1995, JIS-K7350-3:1996, JIS-K7350-4:1996 or a method referring to those.

The light stability in the packaging or container application can be determined by the method of JIS-K7105 and a method referring to that. Typical examples thereof include the light transmittance and transparency evaluation of the bottle body and the functional test of the bottle content after ultraviolet irradiation by using a xenon light source described in JP-A-2006-298456; the haze value evaluation after xenon lamp irradiation described in JP-A-2000-238857; the haze value evaluation by using a halogen lamp as the light source described in JP-A-2006-224317; the yellowing evaluation after mercury lamp irradiation by using a blue wool scale described in JP-A-2006-240734; the haze value evaluation by using Sunshine Weather Meter and the visual observation of color development described in JP-A-2005-105004 and JP-A-2006-1568; the ultraviolet light transmittance evaluation described in JP-A-7-40954, JP-A-8-151455, JP-A-10-168292, JP-A-2001-323082, and JP-A-2005-146278; the ultraviolet-blocking evaluation described in JP-A-9-48935 and 9-142539; the light transmittance evaluation described in JP-A-9-241407, JP-A-2004-243674, JP-A-2005-320408, JP-A-2005-305745, and JP-A-2005-156220; the evaluation of the viscosity of the ink in ink container described in JP-A-2005-178832; the light transmittance evaluation, the visual observation of the container sample and the color difference ΔE evaluation after sunlight irradiation described in JP-A-2005-278678; the ultraviolet light transmittance evaluation, the light transmittance evaluation, and the color difference evaluation after white fluorescent lamp irradiation described in JP-A-2004-51174; the light transmittance evaluation, the haze value evaluation, and the color tone evaluation described in JP-A-2004-285189; the yellowness index evaluation described in JP-A-2003-237825; the light-blocking evaluation and the brightness evaluation by using the color difference Formula of the L*a*b* color system described in JP-A-2003-20966; the yellowing evaluation by using the color difference ΔEa*b* of a sample after irradiation of xenon lights of different in wavelength described in JP-A-2002-68322; the ultraviolet absorbance evaluation after ultraviolet light irradiation described in JP-A-2001-26081; the film tensile elongation test after photoirradiation by using Sunshine Weather Meter described in JP-A-10-298397; the antimicrobial evaluation after photoirradiation in a xenon weather meter described in JP-A-10-237312; the evaluation of discoloration of a package content after fluorescent lamp irradiation described in JP-A-9-239910; the evaluation of oil peroxide value and color tone after fluorescent lamp irradiation of a salad oil-filled bottle described in JP-A-9-86570; the evaluation of the difference in absorbance after chemical lamp irradiation described in JP-A-8-301363; the evaluation of surface glossiness retention rate and appearance after photoirradiation by using Sunshine Weather Meter described in JP-A-8-208765; the evaluation of color difference and bending strength after photoirradiation by using Sunshine Weather-O-meter described in JP-A-7-216152; the light-blocking rate evaluation and the evaluation of the peroxide generated in kerosene described in JP-A-5-139434; and the like.

The long-term durability thereof when the polymer material is used in the coating and coat film applications can be evaluated according to the method of JIS-K5400, JIS-K5600-7-5:1999, JIS-K5600-7-6:2002, JIS-K5600-7-7:1999, JIS-K5600-8:1999, or JIS-K8741 or a method referring to those. Typical examples thereof include the evaluation of the color density, the color difference ΔEa*b* in the CIE L*a*b* color coordinates, and the residual brilliance after photoirradiation in an xenon light-endurance test machine and an UVCON apparatus described in JP-T-2000-509082; the absorbance evaluation after photoirradiation on a film placed on a quartz slide in an xenon arc light-endurance test machine and the evaluation of the color density and the color difference ΔEa*b* in the CIE L*a*b* color coordinates after fluorescent or UV lamp irradiation on wax described in JP-T-2004-520284; the color tone evaluation after photoirradiation in a Metalweather weather-resistance test machine described in JP-A-2006-160847; the evaluation of brilliance retention rate and color difference ΔEa*b* after photoirradiation test by using a metal HID lamp, and the evaluation of glossiness after photoirradiation by a sunshine carbon arc light source described in JP-A-2005-307161; the evaluation by using color difference ΔEa*b*, the brilliance retention rate evaluation and the appearance evaluation after photoirradiation in a Metalweather weather-resistance test machine described in JP-A-2002-69331; the brilliance retention rate evaluation after photoirradiation by using Sunshine Weather-O-Meter described in JP-A-2002-38084; the evaluation by using the color difference ΔEa*b* and the brilliance retention rate evaluation after photoirradiation in a QUV weather-resistance test machine described in JP-A-2001-59068; the brilliance retention rate evaluation after photoirradiation by using Sunshine Weather-O-Meter described in JP-A-2001-115080, JP-A-6-49368, and JP-A-2001-262056; the evaluation of post-irradiation appearance after photoirradiation on a coated plate by using Sunshine Weather-Meter described in JP-A-8-324576, JP-A-9-12924, JP-A-9-169950, JP-A-9-241534, and JP-A-2001-181558; the evaluation of the brilliance retention rate and the change in brightness after photoirradiation by using Sunshine Weather-O-Meter described in JP-A-2000-186234; the evaluation of the appearance of the deteriorated coated film after dew cycle WOM photoirradiation on coated film described in JP-A-10-298493; the evaluation of the ultraviolet light transmittance of coated film described in JP-A-7-26177; the evaluation of the ultraviolet-blocking rate of coated film described in JP-A-7-3189 and JP-A-9-263729; the comparative evaluation of the period until the brilliance retention rate of the coated film declines to 80% by using Sunshine Weather-Meter as described in JP-A-6-1945; the evaluation of rusting after photoirradiation by using a Dewpanel Light Control Weather Meter described in JP-A-6-313148; the evaluation of the strength of a concrete to the coated formwork after external exposure described in JP-A-6-346022; the evaluation by using the color difference ΔEa*b*, the lattice adhesion test and the surface appearance evaluation after external photoirradiation described in JP-A-5-185031; the brilliance retention rate evaluation after external photoirradiation described in JP-A-5-78606; the evaluation of post-irradiation yellowing (ΔYI) by using a carbon arc light source described in JP-A-2006-63162; and the like.

The light stability when the polymer material is used in the ink application can be determined by the method of JIS-K5701-1:2000, JIS-K7360-2, or ISO105-B02 or a method referring to those. Specific examples thereof include the evaluation of the color density and the measurement by the CIE L*a*b* color coordinates after photoirradiation by using an office fluorescent lamp or a discoloration tester described in JP-T-2006-514130; the electrophoretic evaluation after ultraviolet light irradiation by using an xenon arc light source described in JP-A-2006-22300; the print concentration evaluation with a xenon fade meter described in JP-A-2006-8811; the ink blurring evaluation by using a 100 W chemical lamp described in JP-A-2005-23111; the evaluation of the dye residual ratio in the image-forming range by using a weather meter described in JP-A-2005-325150; the evaluation of print chalking and discoloration by using an Eye Super UV Tester described in JP-A-2002-127596; the evaluation of print by using the color difference ΔEa*b* in the CIE L*a*b* color coordinates after photoirradiation by a xenon fade meter described in JP-A-11-199808 and JP-A-8-108650; the reflectance evaluation after photoirradiation by using a carbon arc light source described in JP-A-7-164729; and the like.

The light stability of the solar cell module can be determined according to the method of JIS-C8917:1998 or JIS-C8938:1995 or a method referring to those. Specific examples thereof include the I-V-measuring photovoltaic efficiency evaluation after photoirradiation by a xenon lamp light source having a sunlight-simulating compensation filter described in JP-A-2006-282970; and the evaluation of discoloration gray scale degree, color, and apparent adhesiveness after photoirradiation by using Sunshine Weather Meter or a fade mater described in JP-A-11-261085 and JP-A-2000-144583.

The light stability of fibers and fiber products can be evaluated according to the method of JIS-L1096:1999, JIS-A5905:2003, JIS-L0842, JIS-K6730, JIS-K7107, DIN75.202, SAEJ1885, SN-ISO-105-B02, or AS/NZS4399 or a method referring to those. Examples thereof include the ultraviolet light transmittance evaluation described in JP-A-10-1587, JP-A-2006-299428, and JP-A-2006-299438; the blue scale discoloration evaluation after photoirradiation by using a xenon light source or a carbon arc light source described in JP-A-6-228816, JP-A-7-76580, JP-A-8-188921, JP-A-11-247028, JP-A-11-247027, JP-A-2000-144583, JP-A-2002-322360, JP-A-2003-339503, and JP-A-2004-11062; the UV-blocking rate evaluation described in JP-A-2003-147617; the ultraviolet-blocking property evaluation described in JP-A-2003-41434; the blue scale discoloration evaluation after dry cleaning and after irradiation by using a carbon arc light source described in JP-A-11-302982; the evaluation of lightness index and color difference ΔE* according to chromaticness index after irradiation by using a Fade-O-meter described in JP-A-7-119036 and JP-A-10-251981; the tensile strength evaluation after photoirradiation by using a UV tester or Sunshine Weather Meter described in JP-A-9-57889, JP-A-9-137335, JP-A-10-1868, and JP-A-10-237760; the total transmission and strength retention evaluation described in JP-A-8-41785 and JP-A-8-193136; the ultraviolet protection factor (UPF) evaluation described in JP-T-2003-528974, JP-T-2005-517822, and JP-A-8-20579; the discoloration gray scale evaluation after irradiation by using a high-temperature fade meter described in JP-A-6-228818, JP-A-7-324283, JP-A-7-196631, and JP-A-7-18584; the appearance evaluation after external photoirradiation described in JP-A-7-289097; the evaluation of yellowness index (YI) and yellowing degree (ΔYI) after ultraviolet irradiation described in JP-A-7-289665; the remission evaluation described in JP-T-2003-528974; and the like.

The light stability of the construction material can be evaluated according to the method of JIS-A1415:1999 or a method referring to that. Specific examples thereof include the surface color tone evaluation after photoirradiation by using Sunshine Weather-O-Meter described in JP-A-2006-266402; the appearance evaluation after irradiation by using a carbon arc light source, the post-irradiation appearance evaluation by using an Eye Super UV Tester, the post-irradiation absorbance evaluation, the post-irradiation chromaticity, the color difference evaluation, the evaluation by using the color difference ΔEa*b* of CIE L*a*b* color coordinates after photoirradiation by using a metal HID lamp light source, and brilliance retention rate evaluation described in JP-A-2004-3191 and JP-A-2006-306020; the evaluation of the change in haze value after photoirradiation by using Sunshine Weather Meter and the elongation retention rate after photoirradiation by using a tensile test machine described in JP-A-10-44352, JP-A-2003-211538, JP-A-9-239921, JP-A-9-254345, and JP-A-2003-211606; the evaluation of ultraviolet transmittance after solvent dip-coating and the visual evaluation of post-irradiation appearance by using an Eye Super UV Tester described in JP-A-2002-161158; the evaluation of brilliance change after a QUV test described in JP-A-2002-226764; the brilliance retention rate evaluation after irradiation by using Sunshine Weather-O-Meter described in JP-A-2001-172531; the evaluation by using the color difference ΔEa*b* after ultraviolet irradiation by using a black light blue fluorescent lamp described in JP-A-11-300880; the evaluation of post-irradiation adhesion retention rate and ultraviolet-blocking property by using a UVCON acceleration test machine described in JP-A-10-205056; the appearance evaluation, the total light transmittance evaluation, the haze change evaluation, and tensile shear adhesive strength evaluation after external exposure (JIS-A1410) described in JP-A-8-207218 and JP-A-9-183159; the evaluation of total light transmittance of the light in the entire wavelength range, the haze evaluation, and the yellowing degree evaluation after irradiation by using a xenon weather meter described in JP-A-8-151457; the evaluation of yellowing degree (ΔYI) and ultraviolet absorbent residual ratio after irradiation by using Sunshine Weather-O-Meter described in JP-A-7-3955; and the like.

The light stability when the polymer material is used in the recording medium application can be evaluated according to the method of JIS-K7350 or a method referring to that. Specific examples thereof include the evaluation of the difference in base color in the printing unit after fluorescent lamp irradiation described in JP-A-2006-167996; the evaluation of image density residual rate after irradiation by using a xenon weather meter described in JP-A-10-203033 and JP-A-2004-181813; the evaluation of the change in reflection density after irradiation by using a xenon weather meter described in JP-A-2002-207845; the yellowing degree evaluation based on the L*a*b* evaluation system after irradiation by using a Santest CPS photodiscoloration tester described in JP-A-2003-266926; the post-irradiation discoloration evaluation by using a fade meter described in JP-A-2003-145949; the visual evaluation of post-irradiation discoloration by using a xenon fade meter described in JP-A-2002-212237; the color density retention rate evaluation after indoor sunlight irradiation and the post-irradiation color density retention rate evaluation by using a xenon weather meter described in JP-A-2002-178625; the evaluation of post-exposure C/N by using a fade meter described in JP-A-2002-367227; the fog density evaluation after fluorescent lamp irradiation described in JP-A-2001-249430; the optical reflection density evaluation and the erasability evaluation after irradiation by using a fluorescent lamp described in JP-A-9-95055; the evaluation of post-irradiation color difference ΔE* by using an Atlas fade meter described in JP-A-9-309260; the visual evaluation of post-irradiation discoloration by using a carbon arc fade meter described in JP-A-8-258415; the evaluation of the retention rate of organic EL element color-changing property described in JP-A-2000-223271; the measurement and evaluation of organic EL display brightness after photoirradiation by a xenon discoloration tester described in JP-A-2005-189645; and the like.

Other evaluation methods include those of JIS-K7103 and ISO/DIS9050 or a method referring to those. Specific examples thereof include the appearance evaluation after irradiation of a polycarbonate coating film by a UV tester described in JP-A-2006-89697; the blue scale evaluation after irradiation of a synthetic hair with ultraviolet light described in JP-A-2006-316395; the evaluation of water contact angle on a test cloth after irradiation by using an accelerated weather-resistance test machine described in JP-A-2006-335855; the evaluation of a visual image projected on a projection screen after irradiation by using a weather-resistance test machine described in JP-A-2005-55615; the evaluation of the deterioration of sample surface and visual evaluation of appearance after irradiation by using a Sunshine Weather Meter or a metal weather meter described in JP-A-2005-74735; the visual evaluation of appearance after photoirradiation by using a metal lamp reflector described in JP-A-2005-326761; the evaluation of the light transmittance of bottle label described in JP-A-2002-189415 and JP-A-2004-352847; the evaluation of polypropylene deterioration after irradiation by using a xenon weather meter under humid condition described in JP-A-2003-19776; the evaluation of the deterioration of a hard-coat film by using Sunshine Weather-O-Meter, and the deterioration evaluation, the hydrophilicity evaluation and the abrasion resistance evaluation of the base material described in JP-A-2002-36441 and JP-A-2003-25478; the evaluation of the gray scale color difference of synthetic leather after irradiation by using a xenon lamp light described in JP-A-2003-239181; the evaluation of liquid crystal device characteristics after irradiation by using a mercury lamp described in JP-A-2003-253265; the post-irradiation adhesiveness evaluation by using Sunshine Weather-O-Meter described in JP-A-2002-307619; the evaluation of the degree of turf purpura described in JP-A-2002-293706; the evaluation of ultraviolet light transmittance and tensile strength after irradiation by using a xenon arc light source described in JP-A-2002-114879; the concrete adhesion velocity evaluation described in JP-A-2001-139700; the appearance evaluation and the coated-film adhesiveness evaluation after irradiation by using Sunshine Weather-O-Meter described in JP-A-2001-315263; the evaluation of post-irradiation yellowing degree and adhesiveness by using a carbon arc light source described in JP-A-2001-214121 and JP-A-2001-214122; the adhesiveness evaluation by using a ultraviolet fade meter described in JP-A-2001-207144; the evaluation of insect-repellency when illumination is turned on described in JP-A-2000-67629; the evaluation of the laminated glass yellowing degree (ΔYI) by using an Eye Super UV Tester described in JP-A-10-194796; the evaluation of the surface appearance and brilliance retention rate after QUV irradiation and humidity-resistance tests described in JP-A-8-318592; the evaluation of color difference over time by using a dew panel light control weather meter described in JP-A-8-208976; the evaluation of the glossiness (DI) and the yellowness index (YI) in the wood base-coated state after irradiation by using a xenon Weather-O-meter described in JP-A-7-268253; the ultraviolet absorbance evaluation after repeated processing of UV irradiation and storage in dark described in JP-T-2002-543265 and JP-T-2002-543266; the evaluation of dye discoloration color difference ΔE* after ultraviolet irradiation described in JP-T-2004-532306; and the like.

According to the present invention, it is possible to provide an ultraviolet absorbent that is excellent in production suitability when kneaded with a polymer, or dissolved in a solvent; that is able to not only enhance ultraviolet resistance of a polymer material used together with the ultraviolet absorbent, but also inhibit decomposition of other unstable compounds by usage of the polymer material as an ultraviolet filter; and that is excellent in light fastness while maintaining long-wavelength ultraviolet absorption capacity for a long period of time without both precipitation and bleed-out.

The ultraviolet absorbent of the present invention is excellent in both a long-wavelength ultraviolet absorption capacity and light fastness, so that the ultraviolet absorbent is able to maintain the above-described absorption capacity for a long period of time. Further, the ultraviolet absorbent is also excellent in transparency, and when used in a polymer material, does not color the polymer material. In addition, the ultraviolet absorbent is also superior in convenience in handling, as it has a structure not irritant to the skin. Further, it is possible to incorporate the ultraviolet absorbent in a polymer material by kneading with a polymer substance, or dissolving in a solvent. Further, neither precipitation of the ultraviolet absorbent nor the bleed-out owing to a long-term use of the ultraviolet absorbent occurs in the produced polymer material.

The polymer material of the present invention has advantageous effects that it is excellent in production suitability when kneaded the ultraviolet absorbent with the polymer or dissolved the ultraviolet absorbent in a solvent: it causes neither precipitation of the ultraviolet absorbent nor the bleed-out owing to a long-term use; it is not colored by the ultraviolet absorbent; and it is excellent in both a long-wavelength ultraviolet absorption capacity and lightfastness (ultraviolet light fastness), while maintaining the above-described absorption capacity for a long period of time.

The polymer material according to the present invention, which has favorable lightfastness, can be used for polymeric molded products such as plastic, containers, coatings, coated films, fibers and construction materials. It can also be used, with its superior long-wavelength ultraviolet absorption capacity, in applications for protection of products sensitive to ultraviolet light, such as filter, packaging material, containers, coating, coated film, ink, fiber, construction material, recording medium, image display device and solar cell cover and also in applications for prevention of decomposition of photo-sensitive compounds.

The polymer material according to the present invention can also be used in the cosmetic application. The cosmetic preparation containing the polymer material according to the present invention has advantageous effects that it is resistant to precipitation or yellowing of the ultraviolet absorbent during production of the cosmetic preparation, superior in long-wavelength ultraviolet absorption capacity and also in retention of the absorption capacity for an extended period of time.

In addition, the compound according to the present invention has favorable effects that it has favorable long-wavelength ultraviolet absorption capacity, is resistant to precipitation or bleeding out when used in the polymer material and effective in improving lightfastness, as described above. Further, the compound can protect UV-sensitive organic materials, especially human and animal skins and hairs, from the damaging action by UV irradiation and is thus favorable as a photoprotecting agent for use in cosmetic products and pharmaceutical preparations for human and animals.

The present invention will be described in more detail based on the following examples, but the invention is not intended to be limited thereto.

Any materials, reagents, amount and ratio of use and operations, as shown in the examples, may appropriately be modified without departing from the spirit and scope of the present invention. It is therefore understood that the present invention is by no means intended to be limited to the specific examples below.

### Examples

### Synthesis Example 1

### (Synthesis of Exemplified Compound 2)

1,2-Diphenylpyrazolidine-3,5-dion in amount of 0.8 g (3.2 mmol) and p-tolualdehyde in amount of 1.0 g (8.3 mmol) were agitated at 100° C. for 30 minutes under the condition of nitrogen flow to prepare a reaction mixture, and then the reaction mixture was cooled to room temperature. Thereafter, 10 ml of ethanol was added to the reaction mixture. The above-specified product was obtained as a yellow solid by a recrystallization treatment (yield: 0.94 g, 83%).

The absorption maximum wavelength of the exemplified compound 2 in ethyl acetate solution was 345 nm, indicating that the compound had long-wavelength ultraviolet absorption capacity.

¹H NMR (CDCl₃): δ 2.45 (3H), 7.15 (2H), 7.3-7.4 (6H), 7.45 (4H), 8.1 (1H), 8.45 ppm (2H)

FAB MS (Matrix: 3-Nitrobenzyl Alcohol) m/z 355 ([M+H]⁺), 354([M]⁺, 100%)

### Synthesis Example 2

### (Synthesis of Exemplified Compound 22)

2-Ethoxycarbonyl-1-phenylpyrazolidine-3,5-dion in amount of 0.8 g (3.2 mmol) and p-anisaldehyde in amount of 1.0 g (8.3 mmol) were agitated at 100° C. for 30 minutes under the condition of nitrogen flow to prepare a reaction mixture, and then the reaction mixture was cooled to room temperature. Thereafter, 10 ml of ethanol was added to the reaction mixture. The above-specified product was obtained as a yellow solid by a recrystallization treatment (yield: 1.08 g, 92%).

The absorption maximum wavelength of the exemplified compound 22 in ethyl acetate solution was 378 nm, indicating that the compound had long-wavelength ultraviolet absorption capacity.

¹H NMR (CDCl₃): δ 1.25 (3H), 3.95 (3H), 4.3 (2H), 7.0 (2H), 7.2-7.5 (5H), 8.1 (1H), 8.6 (2H)

FAB MS (Matrix: 3-Nitrobenzyl Alcohol) m/z 367([M+H]⁺), 366([M]⁺, 100%)

Further, other exemplified compounds can be synthesized by referring to the above-described synthetic methods.

### Example 1

(Preparation of molded plates (Sample Nos. 101 to 118))

One (1) kg of a polymethyl methacrylate resin (PMMA) (Tg: 100 to 110° C.) and 0.1 g of the exemplified compound 1 were agitated in a stainless steel tumbler for 1 hour. The mixture was melted and blended by a vent extruder at 230° C. and extruded into pellets for molding by an ordinary method. The pellets were dried at 80° C. for 3 hours, and then, molded into a molded plate having a thickness of 3 mm (Sample No. 101) by an injection molding machine.

Molded plates of the exemplified compounds 2, 3, 4, 5, 6, 7, 8, 10, 11, 12, 14, 22, 23, 24, 25, 29 and 32 (Sample Nos. 102 to 118) were prepared similarly, except that the exemplified compound 1 was replaced with the exemplified compound 2, 3, 4, 5, 6, 7, 8, 10, 11, 12, 14, 22, 23, 24, 25, 29 or 32.

The absorption maximum wavelength λmax values of the exemplified compounds 1, 2, 3, 4, 5, 6, 7, 8, 10, 11, 12, 14, 22, 23, 24, 25, 29 and 32 in ethyl acetate solution were respectively 356 nm, 345 nm, 375 nm, 368 nm, 314 nm, 331 nm, 332 nm, 324 nm, 316 nm, 346 nm, 346 nm, 328 nm, 378 nm, 345 nm, 404 nm, 377 nm, 322 nm and 380 nm.

### (Preparation of Molded Plates (Sample Nos. 119 and 120))

Molded plates of compounds X and Y for comparison (Sample Nos. 119 and 120) were prepared similarly, except that the exemplified compound 1 was replaced with the compound X or Y for comparison. The λmax values of the compounds X and Y for comparison in ethyl acetate solution were respectively 357 nm and 355 nm.

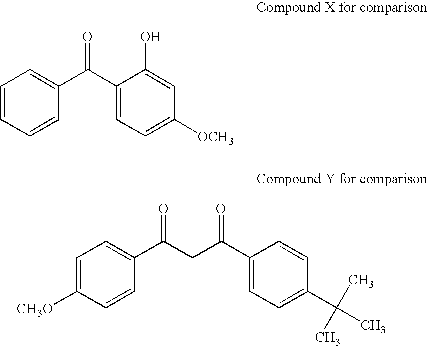

### (Evaluation)

### <Light Fastness Mandatory Test and Wet Heat Toughness Mandatory Test>

Each molded plate prepared was photoirradiated by a xenon lamp with its UV filter removed at an illuminance of 150,000 lux for 100 hours, and the residual amount of the ultraviolet-absorbing compound after irradiation was determined. Molded plates separately prepared in the same manner as the above molded plate samples were allowed to stand for 48 hours under the conditions of temperature 60° C. and relative humidity 80% RH. Thereafter, the residual rate of the ultraviolet-absorbing compound in each of the molded plates was measured. The residual amount was calculated according to the following Formula:

Residual amount (%)=100×(100−Transmittance after irradiation)/(100−Transmittance before irradiation).

The transmittance is a value obtained by measurement at the λmax of the compound added. Results are summarized in Table 2.

TABLE 2

| Sample | Ultraviolet-absorbing | Residual amount (%) after light | Residual amount (%) after wet | |
|---|---|---|---|---|
| No. | compound | fastness mandatory test | heat toughness mandatory test | Remarks |
| 101 | Exemplified Compound 1 | 91 | 94 | This invention |
| 102 | Exemplified Compound 2 | 92 | 92 | This invention |
| 103 | Exemplified Compound 3 | 92 | 93 | This invention |
| 104 | Exemplified Compound 4 | 90 | 95 | This invention |
| 105 | Exemplified Compound 5 | 91 | 94 | This invention |
| 106 | Exemplified Compound 6 | 91 | 96 | This invention |
| 107 | Exemplified Compound 7 | 90 | 90 | This invention |
| 108 | Exemplified Compound 8 | 90 | 91 | This invention |
| 109 | Exemplified Compound 10 | 93 | 88 | This invention |
| 110 | Exemplified Compound 11 | 92 | 92 | This invention |
| 111 | Exemplified Compound 12 | 91 | 94 | This invention |
| 112 | Exemplified Compound 14 | 91 | 94 | This invention |
| 113 | Exemplified Compound 22 | 94 | 95 | This invention |
| 114 | Exemplified Compound 23 | 95 | 94 | This invention |
| 115 | Exemplified Compound 24 | 92 | 94 | This invention |
| 116 | Exemplified Compound 25 | 94 | 93 | This invention |
| 117 | Exemplified Compound 29 | 94 | 91 | This invention |
| 118 | Exemplified Compound 32 | 90 | 86 | This invention |
| 119 | Compound X for comparison | 70 | — (a) | Comparative Example |
| 120 | Compound Y for comparison | 36 | — (a) | Comparative Example |

(a) Beyond measurement owing to white turbidity of the sample

As shown in Table 2, as the results of light fastness mandatory test, the sample Nos. 119 and 120 containing the compound X or Y for comparison had low residual rate of the ultraviolet absorbent after photoirradiation for 100 hours and were thus inferior in lightfastness. In contrast, while each of the sample Nos. 101 to 118 containing the ultraviolet-absorbing compound represented by formula (1) or (2) retained its ultraviolet absorbent in an amount of 90% or more even after photoirradiation for 100 hours, indicating its excellent lightfastness.

As the results of wet heat toughness mandatory test, white turbidity caused by bleed-out was observed in both the sample Nos. 119 and 120 containing the compound X or Y for comparison. In contrast, such white turbidity was not observed in the sample Nos. 101 to 118 each containing the ultraviolet absorbent comprising the compound represented by the formula (1) or (2) of the present invention. In addition, each of these samples had a high transparency. Further from the results of the wet heat toughness mandatory test, it was found that the ultraviolet absorbents comprising the compound represented by the formula (1) or (2) of the present invention was retained in the residual rate of 85% or more, so that they were also excellent in wet heat toughness.

The results show that the polymer material according to the present invention is superior in long-wavelength ultraviolet absorption capacity and also in lightfastness, as the absorption capacity is retained for an extended period of time.

### Example 2

### (Preparation of PET Film Sample No. 201)

A transparent coating consisting of 100 g of DIANAL LR-1065 (trade name, manufactured by Mitsubishi Rayon, 40% methylethylketone (MEK) solution of an acrylic resin) and 0.5 g of the exemplified compound 4 was applied on a 100-μm polyethylene terephthalate (PET) film to be a dry film thickness of approximately 30 μm with a bar coater, and dried to give a PET film sample No. 201 having an ultraviolet-absorbing layer. The absorption maximum wavelength λmax value of the exemplified compound 4 in ethyl acetate solution was 368 nm, indicating that the compound had long-wavelength ultraviolet absorption capacity.

### (Preparation of PET Film Sample No. 202)

A PET film sample No. 202 was prepared similarly, except that the exemplified compound 4 was replaced with the compound Y for comparison.

### (Preparation of PET Film Sample No. 203)

A PET film sample No. 203 was prepared similarly, except that the exemplified compound 4 was replaced with the exemplified compound 22.

### (Evaluation)

A solid image in magenta color was printed on an inkjet-recording paper and dried sufficiently by using an inkjet printer (PIXUS iP1500, trade name, manufactured by Canon), and the PET film prepared above was placed and fixed thereon as an ultraviolet-absorbing layer of the outermost layer. The film was adhered to a southward window glass with its PET film facing the light and left as it was for 12 weeks for a light-resistance test.

Significant discoloration was confirmed in the sample No. 202 having the ultraviolet-absorbing layer containing the compound Y for comparison by visual observation. In contrast, the sample Nos. 201 and 203 having the ultraviolet-absorbing layer containing the exemplified compound 4 or 22 retained a color tone almost similar to that immediately after printing. The facts mean that the polymer material according to the present invention containing the ultraviolet absorbent comprising the compound represented by formula (1) or (2) is also excellent as an ultraviolet-absorbing film for protection of a light-labile compound for an extended period of time.

### Example 3

### (Preparation of Kneaded Ultraviolet Absorbent-Containing Polymer Film (Sample Nos. 301 to 302))

The exemplified compound 3 or 22 in an amount of 15 mg was added to 5 g polyethylene terephthalate in the preparation of a 50 μm film so as to be an absorbance of 1.0 at the absorption maximum wavelength, and the mixture was melt-kneaded at 265° C. and cooled, to give ultraviolet absorbent-containing polyethylene terephthalate films. The ultraviolet absorbent-containing polyethylene terephthalate films were stretched at 280° C. Thereby, ultraviolet absorbent-containing polymer film sample Nos. 301 and 302 were prepared.

In the sample Nos. 301 and 302 employing the exemplified compound 3 or 22, the crystal melted in a short period of time without residual unmelted grains, easily giving a homogeneous and highly transparent sample.

### (Preparation of Kneaded Ultraviolet Absorbent-Containing Polymer Film (Sample No. 303))

An ultraviolet absorbent-containing polymer film (Sample No. 303) was prepared similarly, except that the exemplified compound 3 was replaced with the compound Y for comparison. In the sample No. 303 containing the compound Y for comparison, whiting was observed due to bleed-out.

### (Measurement of Absorption Spectrum)

Absorption spectra of the thus-prepared ultraviolet absorbent-containing polymer film sample Nos. 301 and 302 were measured using a spectrophotometer (UV-3100, trade name, manufactured by Shimadzu Corporation). The results are shown in Table 1.

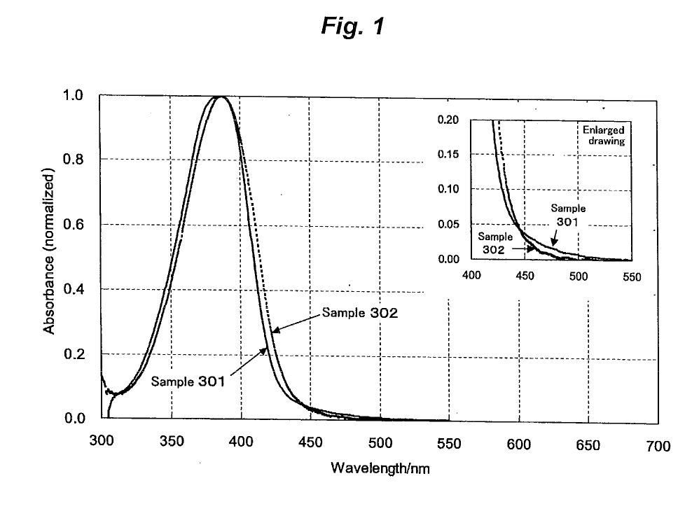

From the results of FIG. 1, it was found that the absorption maximum wavelength was in the long-wavelength ultraviolet range (386 nm and 387 nm) even in a polymer film of polyethylene terephthalate, and therefore the polymer film was useful as an ultraviolet absorbent-containing polymer film capable of shielding a long-wavelength ultraviolet with high efficiency.

Further, it was found that the sample No. 302 containing the ultraviolet absorbent comprising the compound represented by formula (2) (the exemplified compound 22) was excellent in a downward curve of the foot at the long wavelength side end portion at the neighbor of wavelength range of 450 nm to 550 nm, at which range human sensitivity is especially high, compared to the sample No. 301 containing the ultraviolet absorbent comprising the compound represented by formula (1) (the exemplified compound 3). In view of the above, it is understood that despite the ultraviolet absorbent comprising the compound represented by formula (2) has absorption only at a limited long-wavelength portion of the long-wavelength region, the compound gives a colorless and highly transparent sample with almost no coloring, and therefore, the ultraviolet absorbent is especially excellent in the above point.

Having described our invention as related to the present embodiments, it is our intention that the invention not be limited by any of the details of the description, unless otherwise specified, but rather be construed broadly within its spirit and scope as set out in the accompanying claims.

What we claim is:

1. An ultraviolet absorbent, comprising a compound represented by formula (1) or (2): 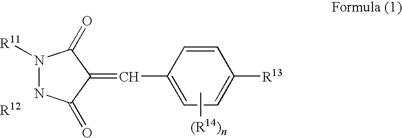
wherein R¹¹, R¹² and R¹⁴ each independently represent a monovalent substituent; R¹³ represents a hydrogen atom or a substituent having a Hammett substituent constant σₚ of −0.35 or more; and n represents an integer of 0 to 4; and 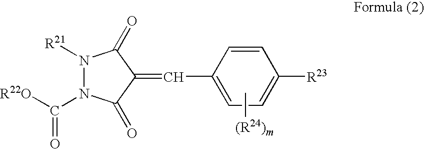
wherein R²¹ represents a substituted or unsubstituted alkyl group having 1 to 18 carbon atoms, or a substituted or unsubstituted phenyl group; R²² represents a substituted or unsubstituted alkyl group having 1 to 18 carbon atoms; R²³ represents a hydrogen atom or a substituent having a Hammett substituent constant σₚ of −0.35 or more; R²⁴ represents a monovalent substituent; and m represents an integer of 0 to 4.

2. The ultraviolet absorbent according to claim 1, wherein, in formula (2), R²¹ is an unsubstituted alkyl group having 1 to 8 carbon atoms, or an unsubstituted phenyl group; R²² is an unsubstituted alkyl group having 1 to 8 carbon atoms; R²³ is a hydrogen atom, an unsubstituted alkyl group having 1 to 8 carbon atoms, or an unsubstituted alkoxy group having 1 to 8 carbon atoms; and m is 0.

3. A polymer material, comprising the ultraviolet absorbent according to claim 1, and at least one kind of polymer substance.

4. The polymer material according to claim 3, wherein the polymer substance is at least one kind of substance selected from the group consisting of acrylic acid-based polymers, polyester-based polymers and polycarbonate-based polymers.

5. The polymer material according to claim 3, wherein the glass transition point (Tg) of the polymer substance is −80° C. or higher and 200° C. or lower.

6. The polymer material according to claim 3, wherein the polymer substance is a polyacrylate, a polycarbonate or a polyethylene terephthalate.

7. The polymer material according to claim 3,
wherein the polymer substance is a polyethylene terephthalate; and
wherein the ultraviolet absorbent is contained in an amount of 0.1 mass % to 50 mass % with respect to 100 mass % of the polyethylene terephthalate.

8. The polymer material according to claim 7, which is prepared by melt-kneading of the polyethylene terephthalate and the ultraviolet absorbent at a temperature of 200° C. or higher.

9. The polymer material according to claim 3,
wherein the polymer substance is a polyacrylate or a polycarbonate; and
wherein the ultraviolet absorbent is contained in an amount of 0.1 mass % to 50 mass % with respect to 100 mass % of the polyacrylate or polycarbonate.

10. The polymer material according to claim 9, which is produced by the steps of:
dissolving the polyacrylate or polycarbonate, and the ultraviolet absorbent in a solvent having a boiling point of 200° C. or lower; and
applying the obtained solution on a substrate.

11. A compound represented by formula (2): 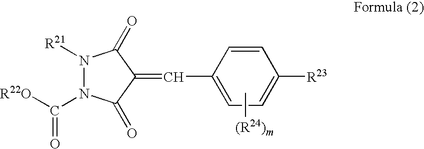
wherein R²¹ represents a substituted or unsubstituted alkyl group having 1 to 18 carbon atoms, or a substituted or unsubstituted phenyl group; R²² represents a substituted or unsubstituted alkyl group having 1 to 18 carbon atoms; R²³ represents a hydrogen atom or a substituent having a Hammett substituent constant σₚ of −0.35 or more; R²⁴ represents a monovalent substituent; and m represents an integer of 0 to 4.

12. The compound according to claim 11, wherein R²³ is a hydrogen atom, a substituted or unsubstituted alkyl group having 1 to 30 carbon atoms, a substituted or unsubstituted aryl group having 6 to 10 carbon atoms, a substituted or unsubstituted alkoxy group having 1 to 30 carbon atoms, a substituted or unsubstituted aryloxy group having 6 to 30 carbon atoms, a substituted or unsubstituted alkoxycarbonyl group having 2 to 30 carbon atoms, a substituted or unsubstituted aryloxycarbonyl group having 7 to 11 carbon atoms, a substituted or unsubstituted carbamoyl group having 3 to 21 carbon atoms, a substituted or unsubstituted acyl group having 2 to 22 carbon atoms, a halogen atom, a hydroxyl group, a cyano group, or a sulfo group.

13. The compound according to claim 11, wherein R²¹ is an unsubstituted alkyl group having 1 to 8 carbon atoms, or an unsubstituted phenyl group; R²² is an unsubstituted alkyl group having 1 to 8 carbon atoms; R²³ is a hydrogen atom, an unsubstituted alkyl group having 1 to 8 carbon atoms, or an unsubstituted alkoxy group having 1 to 8 carbon atoms; and m is 0.
